(19) United States
(12) Patent Application Publication
(10) Pub. No.: US 2006/0183712 A1
(43) Pub. Date: Aug. 17, 2006

(54) Affinity purified heparin/heparan sulfate for controlling the biological activity of the FGF receptor

(75) Inventors: Wallace L. McKeehan, Bellaire, TX (US); Yongde Luo, Pearland, TX (US)

Correspondence Address: HOWREY LLP;C/O IP DOCKETING DEPT., 2941 FAIRVIEW PARK DRIVE SUITE 200, FALLS CHURCH, VA, 22042-2924, US

(73) Assignee: The Texas A&M University System

(21) Appl. No.: 11/060,125

(22) Filed: Feb. 17, 2005

(51) Int. Cl. A61K 31/727

(52) U.S. Cl. 514/56

(57) ABSTRACT

Methods and compositions for modulating the activity of a FGF receptor in a mammal are disclosed. The methods and compositions utilize substantially purified heparin/heparan sulfate oligosaccharides (HS) that have high affinity for FGF7. HS that has high affinity for FGF7 has increased activity for promoting the formation of a ternary FGF/HS/FGFR complex. According to one embodiment of the invention, substantially purified HS is an octasaccharide having 7 or 8 sulfates and having predominant disaccharide composition of ΔHexA2SGlcN6S and a tri-sulfated disaccharide. Alternatively, the substantially purified HS is a longer oligosaccharide that contains an octasaccharide structural motif having 7 or 8 sulfates and having predominant disaccharide composition of ΔHexA2SGlcN6S and a tri-sulfated disaccharide.

### CROSS REFERENCE TO RELATED APPLICATIONS

This application is a non-provisional of Provisional Application Ser. No. 60/345,377, filed Feb. 18, 2004, the entire contents of which are incorporated herein by reference

### FIELD OF THE INVENTION

The invention relates generally to molecular biology and, more specifically, to methods and compositions for modulating the activity of a FGF receptor in a mammal. In particular, affinity-purified heparin/heparan sulfate (herein collectively referred to as HS) oligosaccharides for modulating a FGF receptor activity and a method for obtaining such affinity-purified HS oligosaccharides is disclosed.

### BACKGROUND OF THE INVENTION

Heparin is a highly sulfated free form glycosaminoglycan that exists in the intracellular granule of mast cells. Heparan sulfate is a less sulfated glycan part of proteoglycan molecules that are distributed on the cell surface and are important structural and functional components of the extracellular matrix of all mammalian cells. Heparin and heparan sulfate are linear, polydisperse, highly negative-charged polysaccharide chains composed of alternating uronate and hexasamine saccharides joined by (1--->4) glycosidic linkage. They have a molecular weight range from about 4000 to about 30000 Da.

Heparin has been widely employed as an anticoagulant and antithrombotic drug. The anticoagulant action of heparin resides in its interaction with antithrombin III via a specific pentasaccharide sequence which in turn accelerates the binding and inhibitory activity of antithrombin toward the serine proteases, thrombin, and Factor Xa in the coagulation cascade (Olson, S. T. and Bjork, I., Adv. Exp. Med. Biol., (1992). 313, 155-65; Olson, S. T. and I. Bjork Semin. Thromb. Hemost., (1994) 20(4), 373-409; Olson, S. T., et al., J. Biol. Chem., (1992) 267(18), 12528-38).

Heparin has other beneficial uses in addition to its anticoagulant activity. Examples include treating inflammatory lesions and ischemia/reperfusion (I/R) injury syndromes in pulmonary and myocardial infarction, stroke, traumatic shock, thrombolytic therapy or solid organ transplantations and operations; treating airway allergenic bronchoconstriction or bronchial asthma; treating hemorrhagic, hypovolemic, septic shock and related syndromes; treating atherosclerosis and cancer metastasis; and treating viral infection and wound healing, treating diseases of hypo- or hyper-plasia of tissues such as psoriasis related to the activity of HS-binding proteins such as the FGFs. The non-anticoagulant effects of heparin protects microvascular structures against degradation, preserves myocardial contractility, and the function of heart, lung, liver, gastrointestinal tract, skin and kidney, reduces brain injury and improves immune function, outcome of cancer treatment and regeneration of damaged tissues.

Heparin is isolated from porcine or bovine mucosa or bovine lung tissue for medicinal use. HS are very heterogeneous because of the complexity and nature of their biosynthetic pathway. Further, the composition of HS varies significantly depending on the cellular source and stages of growth and development. The biological activity of HS varies with its homogeneity. For example, less than about 30% of the isolated heparin bears the specific pentasaccharide sequence necessary to interact with antithrombin. The rest of the heparin has essentially no anticoagulant activity. The active part of single heparin molecules of differing lengths is surrounded by large areas of less or different activity.

The use of such heterogeneous heparin as a medicament has been linked to side effects such as hemorrhagic complications, thrombocytopenia, alopecia, osteoporosis, and adverse lipolysis. As many as half of patients receiving heparin for a period over 6 months develop clinically significant osteoporosis. Essentially all patients treated with heparin exhibit a transient thrombocytopenia, and approximately 5% of those patients persist in that state for the duration of therapy. These side effects of heparin significantly limit the clinical use of this important anticoagulant, particularly for long-term use.

Many heparin derivatives aimed at overcoming the problems have been investigated. Low Molecule Weight Heparin (LMWM), obtained by depolymerization and fractionation of unfractionated crude heparin has a lower average molecular weight (4000-6000 Da) and is claimed to have improved properties over unfractionated heparin. These include higher antithrombotic/antihemostatic ratio, higher bioavailability from injection site, longer duration of effect, lower propensity to bind acute phase plasma proteins as well as macrophage and the vascular endothelium and many other tissue proteins, and reduced side effects (Lane D., 1989, London: Edward Arnold; Barrowcliffe, T. W., E. A. J., Duncan P. Thomas, 1992, New York: John Wiley & Sons Ltd). However, these claimed improved properties are still controversial because, although the molecular weight is within quite narrow range, the composition of LMWH is still complicated. There is therefore a strong motivation to develop methods of isolating the biologically relevant portion of HS from among mixtures of HS.

U.S. Pat. No. 6,812,221 (issued Nov. 2, 2004) relates to a method and apparatus for isolating anticoagulant heparin and/or heparan sulfate by binding the anticoagulant heparin or anticoagulant heparan sulfate onto an affinity matrix and separating the non-bound material from the bound material. The affinity matrix is made of a fibroblast growth factor immobilized on a support.

U.S. Pat. No. 5,034,520 (issued Jul. 23, 1991) relates to oligosaccharides composed essentially of chains: possessing a specific affinity for the anionic and cationic cell growth factor which recognize heparin, comprising at least one sequence of 5 residues matching those present in naturally occurring heparin and possessing a strongly anionic character.

Fibroblast growth factors (FGFs) are a family of structurally related polypeptides involved in a number of biological functions including cell growth, differentiation, migration, tissue angiogenesis, wound healing, neurite-outgrowth, organ morphogenesis and development. Aberrant FGF expression is central to progression of many diseases states. Currently 22 human FGFs have been identified, which have a conserved core region of approximately 120 amino acids with 30-70% sequence homology. The FGF family members bind HS with a variable degree of affinity. It has been documented that HS is not only important for the lifetime, storage, protection and confinement of bio-activities of FGFs, but is also indispensable for the regulation of FGFs activity for binding to its cognate receptors (FGFRs). This leads to the formation of an active ternary complex that oligomerizes and is crucial for subsequent intracellular signaling, which then serves as an intrinsic sensor of environmental perturbation and mediators of cell-to-cell and cell-to-environment communication.

Of the 22 distinct FGF homologues, FGF7 (also called keratinocyte growth factor or KGF) exhibits unique functional characteristics that have made it of interest as a clinical pharmaceutical agent. These include an expression pattern largely restricted to the stromal compartment of parenchymal tissues and specificity for a complex of specific heparan sulfate and the FGF receptor isoform, FGFR2IIIb, which is expressed in epithelial cells. FGF7 mediates directionally specific instruction from stroma to epithelium in maintenance of homeostasis of both stromal and epithelial compartments. Since FGF7 impacts proliferation and differentiation in parenchymal epithelial cells of differentiated tissues, it has been proposed for treatment of pathologies associated with dermal adnexae, liver, lung and the gastrointestinal tract diseases, and associated with medical treatments, particularly wound healing in general and diseased conditions of epithelium compartments in various tissues and organs.

U.S. Pat. No. 5,965,530 (issued Oct. 12, 1999) relates to the discovery that KGF (FGF7) stimulates proliferation, growth and differentiation in various cells of epithelial tissue, besides keratinocytes. According to the inventors of the '530 patent, this better understanding of the biological effects of KGF in vivo enables the use of this polypeptide as a therapeutic agent, suitably formulated in a pharmaceutical composition, for the specific treatment of disease states and medical conditions afflicting tissues and organs such as the dermal adnexae, the liver, the lung, and the gastrointestinal tract.

U.S. Pat. No. 6,183,784 (issued Feb. 6, 2001) relates to a milk product extract composition including a plurality of cell growth stimulating factors (including FGF7), extracted from milk product, in concentrated form. The factors have basic to approximately neutral isoelectric points. The '784 patent discusses cell culture compositions and pharmaceutical or veterinary compositions including the above milk product extract and methods for preparing the same.

U.S. Pat. No. 5,843,883 (issued Dec. 1, 1998) relates to a keratinocyte growth factor fragment, KGF_{dcs1-23}, or an analog thereof that is composed of a portion of an amino acid sequence of mature, full-length keratinocyte growth factor, KGF₁₆₃. The fragment exhibits at least a 2-fold increase in mitogenic activity as compared to a mature, recombinant keratinocyte growth factor, rKGF, but lacks a sequence comprising the first 23 amino acid residues, C-N-D-M-T-P-E-Q-M-A-T-N-V-N-C-S-S-P-E-R-H-T-R- of the KGF₁₆₃ N-terminus. The '883 patent also relates to a DNA molecule encoding KGF_{des1-23}, an expression vector and a transformed host containing the DNA molecule, and a method of producing KGF_{des1-23} by culturing the transformed host. The '883 patent further relates to a conjugate of KGF_{des1-23} and a toxin molecule, and the use thereof for treatment of hyperproliferative disease of the epidermis. The '883 patent further relates to a therapeutic composition containing KGF_{des1-23} and a pharmaceutically acceptable carrier and the use thereof for wound healing purposes.

U.S. Patent Application Publication 2003/016650 A1 pertains to a unit dose composition comprising 0.2 μg/kg to 48 μg/kg of an FGF-2 or an angiogenically active fragment or mutein thereof in a pharmaceutically acceptable carrier. The disclosure discusses a method for treating a human patient for coronary artery disease, comprising administering into one or more coronary vessels or a peripheral vein of a human patient in need of treatment for coronary artery disease a safe and angiogenically effective dose of a recombinant FGF-2, or an angiogenically active fragment or mutein thereof. The single unit dose composition described provides an angiogenic effect in a human CAD patient that lasts six months before retreatment is required. The disclosure is also directed to a method of administration that purportedly optimizes patient's safety. In this embodiment, fluids, heparin and/or rate of infusion all play a role. The disclosure also relates to a pharmaceutical composition comprising a therapeutically effective amount of FGF-2, alone or in combination with heparin, in a therapeutically effective carrier.

U.S. Patent Application Publication 2003/0100492 A1 pertains to a molecule for promoting high affinity binding of a fibroblast growth factor (FGF) to a FGF receptor (FGFR), said molecule being selected from: (i) a recombinant chimeric fusion molecule comprising the extracellular domain of a syndecan or a fragment thereof fused to a tag suitable for proteoglycan purification, said fusion molecule being post-translationally glycosylated to carry at least one chain of a heparan sulfate having at least one highly sulfated domain; (ii) a DNA sequence encoding a chimeric fusion molecule comprising the extracellular domain of a syndecan or a fragment thereof fused to a tag suitable for proteoglycan purification; and (iii) a sugar molecule from a syndecan carrying at least one chain of a heparan sulfate having at least one highly sulfated domain. The compounds may purportedly be used for induction of angiogenesis, bone fracture healing, enhancement of wound healing, promotion of tissue regeneration and treatment of ischemic heart diseases and peripheral vascular diseases.

Application of FGF7 in treatment of mucositis of the mucosal lining of the oral or gastrointestinal tract that results from cancer chemotherapy has recently been approved (Kepivance, Amgen). It has also been studied and reported that other FGFs are potent mitogens, morphogens, trophic factors, survival factors and differentiation inducers toward various types of cells from different tissues and organs. Thus FGF signaling has broad potential in diseases as wound healing resulted from physical, chemical, drug-induced and pathological damage in various tissues, in tissue regeneration, in organ/tissue/cell mass transplantation, implantation, engraftment, preservation and stabilization, in promoting neo-angiogenesis and vascularization, and angiogenesis-related therapy, in promoting neuron outgrowth, in birth and defects control, in anticancer related therapies, and in maintaining and promoting stem cell growth. It is therefore desirable to develop methods of modulating the FGF receptor in mammals.

### SUMMARY OF THE INVENTION

The present invention provides a method of modulating the activity of a FGF receptor in a mammal, comprising providing the mammal with substantially purified HS oligosaccharides. When using HS for this purpose, it is desirable to use only the fraction with the greatest activity toward modulating the FGF receptor bio-activities. Surprisingly, it has been found that the fraction of HS oligosaccharides that has the highest affinity for FGF7 is also the fraction that possesses the highest activity with regard to forming the ternary FGF/HS/FGF receptor complex, thereby modulating the FGF receptor bio-activities. Accordingly, one aspect of the present invention is method of modulating a FGF receptor in a mammal, comprising providing the animal with substantially purified HS oligosaccharides, wherein the substantially purified HS oligosaccharides has high affinity for FGF7.

A further aspect of the invention provides a method of modulating a FGF receptor in a mammal, comprising providing the mammal with a composition comprising substantially purified HS oligosaccharides and a FGF.

A still further aspect of the invention provides a composition for modulating the activity of a FGF receptor in a mammal, the composition comprising substantially purified HS oligosaccharides and a FGF.

A still further aspect of the invention provides a method of obtaining substantially purified HS oligosaccharides that modulates a FGF receptor in a mammal, the method comprising: obtaining an affinity matrix comprising a fibroblast growth factor that preferentially binds to HS oligosaccharides that modulates a FGF receptor in a mammal, contacting the affinity matrix with a mixture comprising HS oligosaccharides, separating the non-bound material from the bound material, and obtaining substantially purified HS oligosaccharides as the bound material. According to a preferred embodiment of the invention, the fibroblast growth factor is FGF7.

### DESCRIPTION OF THE FIGURES

FIG. 1. Correlation of high affinity for FGF7, anticoagulant activity and support of binding of FGF7 to recombinant FGFR2IIIb. Enoxaparin LMWH (4-6 kDa) was applied at 0.14 M NaCl to a Sepharose-GSH-GST-FGF7 affinity column prepared and fractions collected at 0.3 (●), 0.60 (□) and then 1.0 (▪) M NaCl. The indicated amounts of the two latter fractions were compared to the fraction at 0.3 M and unfractionated LMWH (▴) binding in support of ¹²⁵I-FGF7 binding to Sf-9 insect cells expressing FGFR2IIIb. The bound FGF7 was expressed as percent of the peak binding value (100%) that could be achieved with 0.3 μg/ml of the 0.6-1.0 M eluate. 100% binding indicated 6000 cpm of ¹²⁵I-FGF7. Binding in the absence of HS was less than 600 cpm. The material recovered at 0.3 M, 0.6M and 1.0 M represented 35%, 40% and 20% of the LMWH, respectively.

FIG. 2. Generation and purification of oligosaccharide mixtures of defined length. Porcine intestinal mucosal heparin (PIMH) was partially digested by heparinase 1 and oligosaccharides of the indicated lengths in monosaccharide units were separated by gel filtration. Oligosaccharides were monitored by absorbance at 226 nm. A synthetic antithrombin-binding pentasaccharide (AT5) was employed as a standard. V₀ and Vₜ were determined by blue dextran (M.W. 2000 kilodalton) and acetone (M.W. 58 dalton), respectively. The center part of each peak was collected and subjected to the same procedure. Purity of oligosaccharides in respect to length was monitored by gradient PAGE and sugars revealed by Alcian Blue.

FIG. 3. Comparison of anticoagulant and FGF7 binding activity of oligosaccharide mixtures of increasing lengths. (A) Anticoagulant activity. Antithrombin-mediated inhibition of Factor Xa activity of oligosaccharides of the indicated length in monosaccharide units at 0.3 μM was determined spectrophotometrically. Activity is expressed as percent Factor Xa activity in antithrombin and Factor Xa mixtures in absence of heparin (100% activity). AT5, synthetic anticoagulant pentasaccharide. H, unfractionated PIMH. 100% activity represented about 0.54 A405 units. (B) FGF7 binding activity. The same oligosaccharides at 0.3 μM were tested for support of binding of FGF7 to FGFR2IIIb. Amount of bound radiolabeled FGF7 was expressed as a percentage of that bound in the presence of 0.4 μg/ml crude PIMH (H) (100%). 100% binding represented about 6000 cpm of bound ¹²⁵I-FGF7 and less than 600 cpm bound in the absence of added HS.

FIG. 4. Anticoagulant activity of FGF7-affinity purified oligosaccharides. Fractions of the octasaccharide, decasaccharide, dodecasaccharide and tetradecasaccharide mixtures eluted from FGF7 affinity column at the indicated NaCl concentrations (Table 1) were tested for antithrombin-mediated inhibition of Factor Xa at 0.1 μM as in FIG. 3A. Crude indicates the unfractionated mixture applied to the column. Concentrations of PIMH (H) was 0.13 μg/ml and synthetic AT-binding pentasaccharide (AT5) was 0.1 μM. 100% activity represented 0.54 absorbance units at A405.

FIG. 5. Activity of oligosaccharides with graded affinity for FGF7 for support of FGF7 binding to FGFR2IIIb. Activity of the indicated fractions of the octasaccharide, decasaccharide, and dodecasaccharide mixtures from the FGF7 affinity column described in FIG. 4 was assayed at 0.3 μM as described in FIG. 3B. 100% binding activity represented 6000 cpm ¹²⁵I-FGF7 and was the amount of binding supported by crude PIMH (H) at 0.4 μg/ml.

FIG. 6. Confirmation of specific FGF7-FGFR2IIIb complexes supported by FGF7-affinity purified oligosaccharides by covalent affinity crosslinking. Covalent affinity cross-linking analysis with agent DSS was performed on FGFR2IIIb-expressing cells induced to bind ¹²⁵I-FGF7 by 0.3 μM of the octasaccharide and dodecasaccharide fractions from FGF7-affinity column described in FIG. 5 except the concentration of ¹²⁵I-FGF7 was increased from 2 ng/ml to 12 ng/ml. The crosslinked binding mixture was separated on 7.5% SDS-PAGE. The gel was dried and subjected to autoradiography.

FIG. 7. Concentration-dependent activity of octasaccharide fraction in support of the FGF/HS/FGFR ternary complex formation. Effects of the high-affinity fraction (▪), 0.6 M NaCl fraction (▴), unbound fraction (●) and crude octasaccharide (□) from the FGF7 affinity at different concentration on the ternary complex formation were assayed as described in FIG. 3B. The bound FGF7 was expressed as percent radioactivity of peak binding value that can be reached. Data is the representative of at least three independent assays.

FIG. 8. Charge separation and characterization of FGF7-affinity purified octasaccharide fractions. One hundred μg of crude octasaccharide (A), 0-0.14 M NaCl fraction (B), 0.14-0.3 M NaCl fraction (C), 0.3-0.6 M NaCl fraction (D) or 10 μg 0.6-1.0 M NaCl fraction (E) from FGF7 affinity were subjected to anion-exchange chromatography on a Propac PA1 column, eluted by a linear gradient concentration of NaCl solution. The heparin oligosaccharides were detected by UV absorbance at 226 nm.

FIG. 9. MALDI-TOF mass spectrometric analysis. About 11 ng of high affinity octasaccharides (A) or octasaccharides with lower affinity (peak 0306A) (B) (2 μl) for FGF7 was mixed with about 50 ng synthetic peptide carrier [(Arg-Gly)₁₉-Arg] (1 μl) in the presence of 4 μl 15 mg/ml caffeic acid in 40% aqueous acetonitrile. Aliquots (2 μl) of the mixture were deposited on a polished stainless steel chip, dried, and analyzed in a Bruker Autoflex MALDI-TOF mass spectrometer in a linear positive mode with 120 ns delayed extraction and 2000 Da mass gate. Observed in each mass spectrum were the (M+H)⁺ ions of the basic peptide and the (M+H)⁺ ions of a 1:1 peptide/saccharide complex. Asterisk indicates non-specific signal mainly from the degraded species of peptide [(Arg-Gly)₁₉-Arg] and their complex with oligosaccharides.

FIG. 10. Disaccharide compositional analysis of the high-affinity octasaccharides by MALDI-TOF mass spectrometry. The high-affinity fraction was reduced to disaccharides by a combination of heparinase 1, 2 and 3 overnight at 37° C. The MALDI-TOF mass spectrometry analysis of this digestion mixture was done as described in FIG. 9.

FIG. 11. Disaccharide compositional analysis of the high-affinity octasaccharides by strong ion-exchange chromatography. About 4 μg high-affinity fraction of octasaccharides from FGF7 affinity was exhaustively digested by a combination of heparinase 1, 2 and 3 overnight at 37° C. The digestion mixture was desalted by Sephadex G-25 in water and concentrated by centrifugal evaporation. The mixture was subjected to anion exchange chromatography as described in FIG. 8 except for the scheme of elution gradient as described in the Example section. The identified disaccharides were indicated by arrow.

FIG. 12. Disaccharide compositional analysis of the high-affinity octasaccharides by ion-pair reverse phase chromatography. About 4 μg HA fraction of octasaccharides from FGF7 affinity was exhaustively digested by a combination of heparinase 1, 2 and 3 overnight at 37° C. The digestion mixture was desalted by Sephadex G-25 in water and concentrated by centrifugal evaporation. The mixture was subjected to ion-pair reverse phase chromatography, which was conducted on Supercosil LC-18 column in the presence of tetrabutylammonium phosphate. The bound disaccharide was released by a gradient scheme of aqueous acetonitrile, and detected by absorbance at 226 nm as described in the Example section. The identified disaccharides were indicated by arrow.

FIG. 13. Anticoagulant activity of FGF1-affinity purified oligosaccharides. Fractions of the octasaccharide and dodecasaccharide eluted from FGF1 affinity column at the indicated NaCl concentrations (Table 2) were tested for antithrombin-mediated inhibition of Factor Xa at 0.1 μM as in FIG. 3A. Crude indicates the unfractionated mixture applied to the column. Concentrations of PIMH (H) was 0.13 μg/ml and synthetic AT-binding pentasaccharide (AT5) was 0.1 μM. 100% activity represented 0.58 absorbance units at A405.

FIG. 14. Activity of oligosaccharides with graded affinity for FGF1 for support of FGF1 binding to FGFR1IIIc. Activity of the indicated fractions of the octasaccharide and dodecasaccharide mixtures from the FGF1 affinity column described in FIG. 13 was assayed at 0.3 μM as described in FIG. 3B. 100% binding activity represented 6000 cpm ¹²⁵I-FGF7 and was the amount of binding supported by crude PIMH (H) at 0.4 μg/ml.

FIG. 15. MALDI-TOF mass spectrometric analysis. About 11 ng of 0.6-1.0 M (A)and 1.0-1.7 M (B) NaCl fractions of octasaccharides from FGF1 were analyzed in a Bruker Autoflex MALDI-TOF mass spectrometer in a linear positive mode as described in FIG. 9. Observed in each mass spectrum were the (M+H)⁺ ions of the basic peptide and the (M+H)⁺ ions of a 1:1 peptide/saccharide complex. Asterisk indicates non-specific signal mainly from the degraded species of peptide [(Arg-Gly)₁₉-Arg] and their complex with oligosaccharides.

### DETAILED DESCRIPTION OF THE INVENTION

The present invention provides a method of modulating the activity of a FGF receptor in a mammal, comprising providing the mammal with substantially purified heparin/heparan sulfate (HS) oligosaccharides. As used herein, HS refers to heparin, heparin sulfate, mixtures and any natural or semisynthetic or synthetic derivatives thereof. HS's are sulfated linear glycosaminoglycan (GAGs) chains comprising alternating hexuronate and glucosamine residues presented as side-chains of several proteoglycans on the surface and in the extracellular matrix of all adherent cell types in metazoan organisms. HS play fundamental roles in diverse biological events ranging from preservation of critical anticoagulant surface of vascular endothelium and blood system homeostasis, control of cell growth, adhesion and communication, lipid metabolism, to cancer, infection and inflammatory responses. The functions of HS are fulfilled by their incredible ability to interact with and modulate the activity of a variety of growth factors/cytokines/chemokines, receptors, enzymes, proteases, protease inhibitors and lipids. The multiplicity of various interactions relies on the structural heterogeneity of HS chains in respect to their length and chemical composition, as well as the nature of hydrogen bond and ionic interactions with partners. The heterogeneity of HS is largely the results of different degree and positioning of enzymatic sulfation on 2-OH groups of uronic acids, 2-NH₂, 3-OH and 6-OH groups of glycosamine residues, acetylation or unsubstitution on 2-NH₂ groups of glycosamine residues, and epimerization interspersed along HS chains that have varied length, by a battery of endo-enzymes with spatial and temporal expression pattern. To date 24 different types of HS disaccharides out of potentially as many as 48 disaccharides have been experimentally identified, making HS more complicated than DNA with 4 bases and peptide/protein with 20 amino acids as building blocks. Most of the interactions of HS with other molecules are dominated by ionic interactions between negative charges of sulfate and carboxyl groups on HS as primary functional groups and positive charges of side chains of basic amino acids spatially arranged into a stretched shallow pocket on one or more surface domains of a particular protein, as well as the hydrogen bond between the hydroxyl groups on HS and amino groups on a protein. The considerable interest of current studies is whether the interaction requires a specific motif as in the case of the antithrombin interaction with a pentasaccharide motif, or is just random and nonspecific ionic interaction.

There has been an increasing realization that HS displays fine specificity in addition to the incredible diversity for the interaction with different proteins and that these interactions result in the selective modulation of protein activity. The protein binding sites comprise relatively small tracts of disaccharides with different functional groups in specific arrangements; usually 3-9 disaccharides out of 20-80 disaccharides in a small portion of HS chains represent the actual binding sites for ligands. This is thoroughly exemplified by their role in the maintenance of blood homeostasis. HS with a minimal pentasaccharide sequence, GlcN6S-GlcA-GlcNS3S-IdoA2S-GlcNS6S, binds to a blood plasma serpine, antithrombin III (ATIII), with high affinity. The complex formation between pentasaccharide and ATIII results in conformational change of the latter that reveals an active-site loop, allowing ATIII to form an inactive complex with more than three-order of magnitudes increase of reaction rate with the targeted serine proteases in the blood clotting cascade, notably the Factor Xa and Ia (also called thrombin). Only 1-5% of heparan sulfate chains in the vascular endothelium lining contain this motif. The selectivity of ATIII for this short motif over a considerably heterogeneous HS pool is thus remarkable.

The binding of FGFs to HS seems promiscuous, yet evidence from both biochemical and structural studies shown that FGFs actively utilize HS segments with specific arrangement of functional groups for the expression of their optimal biological activities via the formation of an intimately associated ternary complex with each other and with FGFRs. In the absence of HS chain from either endogenous proteoglycan or exogenous addition, FGF is much less active or has no activity toward FGFR. In this sense a short portion of specific composition from HS chain of large heterogeneous pool is definitely needed for initiating, activating and enhancing FGF-mediated FGFR signaling. The other portion of HS is essentially not need for FGF-mediated activity, although may play a role in the storage and stabilization of secreted FGFs. Difference in structure of HS motif not only affects the binding affinity of FGFs to HS, but also the ability of FGFs to activate specific FGFRs for subsequent signaling. Crystallographic and modeling analysis of FGFs revealed common as well as distinct features of composition of HS-binding domains among different FGFs. This underlies the basis of common as well as distinct requirements of FGF activity for HS motif. It has been generally thought that a length encompassing five to seven monosaccharide of HS may be sufficient for binding with significant affinity to FGF1 and FGF2, the prototypes of FGF family, and FGF8b, but a longer length, from octasaccharide to dodecasaccharide with both 2-O— and 6-O— sulfate groups was required for spanning and forming active FGF/HS/FGFR ternary complex. Moreover, it was shown that distinct sulfates with different positioning were required for interaction with FGF1 and FGF2. The binding of FGF1 and FGF8b to HS may require a critical disaccharide containing both IdoA 2-O— sulfate and GlcNS 6-O— sulfate groups, yet the binding of FGF2 needs a single IdoA 2-O-sulfate group, although additional sulfates may enhance the interaction.

When crude HS or their derivatives are applied, the ultimate stimulation of activity of FGF/HS/FGFR complex is the net effect of activating, interfering and even inhibiting motifs residing on the same or different HS chains on the particular FGF/FGFR pair involved. Just as in the case of anti-clotting treatments, one should be cautious when using HS that has tremendous heterogeneity, promiscuity and tendency for non-specific ironic interactions. Thus, a complex formed between a short but specific and productive oligosaccharide and a particular FGF has increased stability, better dual-specificity and much more potent and immediate activity, and increased availability and reduced non-specific and non-productive retention than FGF alone, when applied to patients with a variety of indications. In addition, although purified HS oligosaccharides are applied, they are short and only in a high-affinity bound form to FGF, thus will not cause side-effects associated with crude HS applications. FGFs have potent activities in the presence of HS for promoting growth, proliferation, survival and differentiation of a wide variety of cells and tissues of mesodermal, ectodermal and endodermal origin, including fibroblasts, epithelial cells, endothelial cells, muscle cells, neuronal cells, hematopoietic cells, keratinocytes, osteoblasts, chondrocytes, myoblasts, astrocytes, adipocytes, and oligodendrocytes, etc. They have been continuously pursued as therapeutic agents for a number of different indications, including (1) wound healing caused by musculo-skeletal damages such as bone fractures, ligament, tendonitis, bursitis, etc; by diabetes, ischemic blockage or injury, malnutrition, obesity, infection, chemotherapy, radiation therapy, and immunosuppression, other drug-related damages; (2) skin conditions such as burns, cuts, lacerations, bed sores, ulcers etc, and other organ-related surgical damages; (3) induction of angiogenesis, tissue protection and repair during myocardial infarction, ischemia and stroke; (4) neurological conditions such as neuro-degenerative disease, stroke, and brain and spinal cord injures; (5) congenital defects, fertility or abnormal growth; (6) cancer, and other proliferative, differentiation disorders or damages from neural tissue, skin, placenta, thymus, eye, liver, kidney, pancreas, lung, bladder, prostate, stomach, intestine and esophagus; (7) inflammatory conditions, such as psoriasis, ulcerative colitis and Grohn's disease; and (8) maintaining, promotion and induction of differentiation of stem cells for implanting functioning cells or derived tissue/organ-like mass to patients.

The FGFs that have been utilized or proposed in the various clinical indications stated above were typically administered alone, without regard for HS (the inventions described in U.S. Patent Applications 2003/0100492 and 2003/0166550 included administering HS along with FGF, but did teach or suggest that the HS was in any way enriched with the fraction of HS capable of promoting the ternary FGF/HS/FGFR complex). The FGF's interaction with tissue heparan sulfate and other mimicking polyelectrolytes directs their endpoint biological activity. The FGFs encounter the abundant general extracellular content of reservoir heparan sulfate. In this reservoir are heparan sulfate motifs with a broad range of affinities for FGF. Those with highest affinity have most impact on the biological activities of FGF and FGFR. These high-affinity motifs, if they remain fixed in the tissue matrix, are most effective in controlling access to the FGFR and protecting FGF from degradation to prolong the lifetime of FGF and FGF-stimulated FGFR activity in the harsh biological environment. If they are mobile, they form a tighter, more stable and active complex with FGF, FGFR or FGF/FGFR than low-affinity and inactive motifs. In addition, the ectodomain of the FGFR kinase may be normally complexed to specific heparan sulfate that forms the active site of the FGFR. Such an oligosaccharide motif must be specific for both the FGFR and the activating FGF. Biospecific oligosaccharides of defined structure can be applied together or in a complex with administration of external FGF to enhance its availability and half-life, or alone to where there is sufficient FGF present endogenously without external administration. The heparan sulfate environment may be altered significantly in diseased tissues, where the specific motif for FGF may not exist or exist at insufficient level. This could reduce the life-life and drug efficacy of FGF administered alone, limit or prevent the binding and activation of the FGFR complex by the administered FGF. The short, sized oligosaccharides of specific motif composition of the present invention have the highest affinity and thus specificity to bind FGF, and highest capability to potentiate FGF stability and thus its activity. They also have highest activity to confer FGF with highest potential to activate FGFR, thus highest biological effects. The complex formed between FGF and these high-affinity, high-activity oligosaccharides thus obviously have immediate activity of activating FGFR at lower concentration but full efficacy at longer effective time than FGF administered alone. Since the HS oligosaccharide administered is defined and has relatively short length, and consequently with lower dose than crude higher molecular weight HS, the severe non-specific side effects of crude HS are alleviated.

The present invention utilizes high-affinity, high-activity oligosaccharides extracted by affinity for a specific FGF or FGFR with specific structural motifs from HS. These oligosaccharides are from a small portion of the total HS pool. Typically, they are short and size-defined, and have minimal structure with full activity, so will not disturb the normal functions of tissues, and have less or no side effects of crude HS. The oligosaccharides of the present invention are capable of forming a stable complex with FGF by ionic interaction with highest affinity, so FGF in this complex will have minimal association non-specifically and non-productively with cell surface components other than FGFRs. The oligosaccharides confer FGF with the highest activity possible with highest clinical potential compared to FGF alone or in combination with crude HS.

The present invention provides methods and compositions for modulating a FGF receptor in a mammal using substantially purified HS. Surprisingly, it has been found that the fraction of HS oligosaccharides that has the highest affinity for FGF7 is also the fraction that possesses the highest activity with regard to forming the active ternary FGF/heparin/FGF receptor complex, thereby modulating the FGF receptor in term of both inhibition and activation. Accordingly, one aspect of the present invention is method of modulating the activity of a FGF receptor in a mammal, comprising providing the animal with substantially purified HS, wherein the substantially purified HS has high affinity for FGF7.

An effective method for producing and recovering quality FGFs fused to glutathione-S-transferase in E. coli and an improved method for large-scale production of GST-FGF7 or FGF7 has been disclosed in Luo, Y., H—H Cho, R. B. Jones, and W. L. McKeehan, Improved Yield of Recombinant Fibroblast Growth Factor 7 (FGF7/KGF) from Bacteria in High Magnesium Chloride Protein Expression & Purification (2003) and in U.S. Pat. No. 6,812,221 B (issued Nov. 2, 2004), the entire contents of which are incorporated herein by reference. These methods ensure the quantity and quality of FGF needed for high biological specificity and activity and clinical applications at a low cost.

Crude HS has tremendous heterogeneity, promiscuity and tendency for non-specific ionic interactions. When crude HS or their derivatives are applied, the ultimate stimulation of activity of FGF/HS/FGFR complex is the net effect of activating, interfering and even inhibiting motifs residing on the same or different HS chains on a pair of particular FGF and FGFR involved. According to the present invention, affinity-purification of crude porcine intestinal mucosa heparin (PIMH) and low molecular weight heparin (LMWH) with immobilized GST-FGF7 indicates that specific structural motifs in HS are required for high-affinity binding to FGF7 and for FGFR activation, which may be related to, but different in detail from the anticoagulant motif. Multidimensional chromatography including size exclusion, FGF-affinity, ion exchange and ion-pairing reverse-phase chromatography coupled with mass spectrometric analysis and combined with biological activity assays to isolate the specific FGF7-binding and -activating motif identified a fraction of heparin oligosaccharides of 8 to 14 monosaccharides in length with activities of high-affinity FGF7 binding as well as ability to activate FGFR. The fraction also maintains anticoagulant activity monitored by activation of antithrombin III (ATIII). The isolation and biological use of this particular fraction of HS oligosaccharides are within the scope of subjects of this invention.

The inventors have discovered specific HS oligosaccharides based on the affinity for specific members of the 22 members of the FGF polypeptides or the four FGFR ectodomains have high activity for assembly of the FGF/HS/FGFR ternary complex. The short oligosaccharides of the present invention derived based on such affinity comprise only 1-10% of a starting pool of crude oligosaccharides, however, they elicit almost all the activity for FGF-mediated FGFR activation. The rest of the oligosaccharides in the pool have little or no activity, but may be related to the non-specific binding or retention of FGFs, or general protection of FGF against destabilization or inactivation by various physical, chemical or enzymatic factors, or confinement or sequestration of available FGF activity. The prior art does not teach the utility of such FGF-specific oligosaccharides in formation of an FGF/oligosaccharide complex that has enhanced stability, dual-specificity (both FGF specificity to a particular FGFR and oligosaccharide specificity to a FGF or FGF/FGFR combination) with enhanced activity, better availability and longer half-life. Since the majority of crude HS chain has no specific activity for activation of FGF or FGFR, the use of crude HS may cause severe side effects and actually reduce FGF efficiency administered as a drug because of the dominant non-specific and non-productive binding to FGF or FGFR and other biological factors.

The activity of FGF is controlled by species of HS having specific structures within the bulk class of molecules referred to as HS. Without the assistance of these HS species, FGF is an inactive polypeptide. The normal source of HS is the cell and tissue repertoire with which FGF and FGFR interact. It is known from the available crystal and modeling structures of FGFs that FGFs have a common characteristic HS-binding domain, which mainly comprises side-chains of basic amino acids with positive charge that can attract negative charge of sulfate and carboxylate groups on HS chains. (Ye, S., Luo, Y., Lu, W., Jones, R. B., Linhardt, R. J., Capila, I., Toida, T., Kan, M., Pelletier, H., and McKeehan, W. L. Structural basis for interaction of FGF-1, FGF-2, and FGF-7 with different heparan sulfate motifs. Biochemistry (2001); Faham, S., Hileman, R. E., Fromm, J. R., Linhardt, R. J., and Rees, D. C. Heparin structure and interactions with basic fibroblast growth factor. Science (1996); DiGabriele, A. D., Lax, I., Chen, D. I., Svahn, C. M., Jaye, M., Schlessinger, J., and Hendrickson, W. A. Structure of a heparin-linked biologically active dimer of fibroblast growth factor. Nature (1998); Yeh, B. K., Igarashi, M., Eliseenkova, A. V., Plotnikov, A. N., Sher, I., Ron, D., Aaronson, S. A., and Mohammadi, M. Structural basis by which alternative splicing confers specificity in fibroblast growth factor receptors. Proc Natl Acad Sci USA (2003); Plotnikov A N, Eliseenkova A V, Ibrahimi O A, Shriver Z, Sasisekharan R, Lemmon M A, Mohammadi M. Crystal structure of fibroblast growth factor 9 reveals regions implicated in dimerization and autoinhibition. J. Biol. Chem. (2001)). However, HS-binding domain from different FGFs differs, thus predicting specificity in respect to the HS motifs with which it interacts. The inventors have discovered that HS with different structural motifs have different affinity and activity to different FGF. These phenomena set a basis for isolation and characterization of various HS structures with specific activities.

Crude HS in nature is a class of polyelectrolyte carbohydrate of extremely heterogeneous character with similar backbone structure. Thus it has high capacity for non-specific ionic interactions with positively charged molecules. The non-specific effects of some HS species on target proteins are stimulatory while others are inhibitory or interfering. An extraction according to the affinity to target molecules, such as herein the FGF, FGFR or the ternary complex, is one important approach to separate the stimulatory or inhibitory species from the mixture. It has been well documented that the activity of FGF is dependent on and potentiated by the presence of certain amount of HS, even in a crude form. In fact, immobilized heparin has been a ubiquitous way to purify FGF.

The inventors previously discovered that FGF7, a unique member of the fibroblast growth factor polypeptide family, is able to bind anticoagulant heparin or the LMWH (low molecular weight heparin) fraction with high affinity at more than 50% efficiency of that of antithrombin. FGF7 could be used to separate and pull down anticoagulant fraction from non-anticoagulant part in crude HS or low molecular weight heparin (LMWH) product or drug (Yongde Luo H-H, Cho and Wallace L. McKeehan (2001) Luo, Y., and W. L. McKeehan. (2003) Journal of Pharmaceutical Sciences 92, 2117-2127; U.S. Pat. No. 6,812,221). The results indicated that FGF7 might utilize an anticoagulant-related motif on HS for high-affinity binding.

Surprisingly, the fraction of HS oligosaccharides that has a high affinity for FGF7 shows an enrichment of activity for supporting FGF7 binding to its receptor FGFR211Ib. This indicates that there is a portion of HS oligosaccharide that can be enriched or extracted by FGF7 affinity within crude oligosaccharides that effectively supports FGF7 binding to the receptor in addition to enriching the antithrombin-mediated anti-Factor Xa activity.

The vast majority of HS or LMWHs have chains with length greater than 8-12 monosaccharide units, the minimum range of chains that may elicit biological effect on FGF signaling. A single chain from HS or LMWHs may carry within it multiple motifs with distinct types of activities both specific in regard to impact on the FGF system and non-specific impact on many other processes. The longer length the chain has, the higher the probability that these multiple motifs and activities co-exist. Thus it is important to eliminate the masking effect of the majority of crude HS or LMWH motifs and limit them to structural motifs that impact specific FGF activities. The isolated HS oligomers with optimum homogeneity are valuable for defining structural motifs within HS that is physiologically relevant to the desired activity. The information obtained about a unique structure-activity relationship of an oligosaccharide for a particular FGF and its impact on FGF signaling is important for design and test potential agonists or antagonists of FGF bioactivity based on the structural information.

Accordingly, an aspect of the invention is a method of size-selecting and isolating short oligosaccharides with high activity to modulating FGFR signaling. The size effect of HS on the activities of various proteins has been observed. This is partly due to the increased availability of the array of multiple either same or different active motifs in both the inter- and intra-chains of HS when the chain length increases. However, for structure-activity studies, obtaining shortest chains yet with full activity by size- and affinity-selection facilitates subsequent structural analysis. Contacting crude or LMW HS with a heparinase can provide short oligosaccharides, which can be purified based on FGF affinity. For example, heparinase 1, recognizes the highly sulfated region of and has highest enzymatic activity toward heparin among HS chain-degrading enzymes. Heparinase 1 can be used to partially cleave crude HS mixture with chain length range from about 20 to 60 disaccharides. The resulting mixture can be passed through a column of porous polyacrylamide-based polymer matrix. The presence of HS oligomer can be detected by using UV absorbance. The purity can be determined using gradient PAGE. Although it has been reported that heparinase 1 can recognize and cleave the anticoagulant motif of HS, by controlled partial digestion, a portion of the anticoagulant sequences in HS chains can be preserved.

A mixture of shorter HS oligomers have a similar overall chemical constitution as crude HS or LMWH, but less chance for co-existence of the same or different sequence motifs on a single chain. In addition, some active motifs are destroyed at the site of cleavage. Because of this, reduction of chain length unavoidably reduces the overall activity in the mixture without impact on the nonspecific electrolyte character. This has been demonstrated by LMWHs that LMWHs have overall less anticoagulant activity than its parent materials due to the chemical or enzymatic process used in size reduction that destroys part of the anticoagulant motif.

Reduction of size to short oligomers greatly reduces the ability of HS to accelerate antithrombin-mediated anti-Factor Xa, activity and to support FGF7 binding to FGF receptor (FGFR). Crude heparin disaccharide, tetrasaccharide and hexasaccharide are devoid of both inhibitory and stimulatory activity for Factor Xa and FGF7 binding to FGFR2IIIb respectively at 0.3 μM concentration. At the same concentration, crude but size-uniform octasaccharide and decasaccharide exhibited 30% and 75% inhibitory activity for Factor Xa respectively. Dodecasaccharide and tetradecasaccharide have inhibitory activity close to that of the synthetic antithrombin-binding pentasaccharide, which has over 90% inhibition for Factor Xa activity, or that of 0.4 μg/ml crude porcine intestinal mucous heparin (PIMH). Octa-, deca-, dodeca-, and tetradeca-saccharide have 2%, 24%, 70% and 90% activity for supporting FGF7 binding to FGFR2IIIb at 0.3 μM concentration compared to 100% of 0.4 μg/ml crude PIMH. Crude dodecasaccharide at this concentration may be the minimum size to elicit over 50% activity required in FGF/HS/FGFR complex formation.

An FGF7 affinity matrix can be made by immobilizing GST-FGF7 on GSH-Sepharose column by bioaffinity between GST and GSH partnership, or on NHS-activated Agarose matrix by covalent bond formation. Both immobilization methods preserved the intact heparin-binding ability of GST-FGF7 and FGF7 (data not shown). According to one embodiment of the invention, a FGF affinity matrix is prepared as described in U.S. Pat. No. 6,812,221. High quality GST-FGF7 and FGF7 can be produced in the presence of 30 mM MgCl₂ in BL21 DE3 pLysS bacteria with high yield, as described in the '221 patent. Since HS octasaccharide is the threshold lower limit in size that begins to elicit activities for both inhibiting Factor Xa and supporting FGF7 binding, HS oligomers from octa- to tetradeca-saccharide are typically chosen for affinity-purification and subsequent SAR. NaCl at different concentrations can be used to elute different binding-affinity fractions of HS oligosaccharides from the affinity matrix, and the minimum concentration at which a fraction elutes can be used to differentiate the fraction's affinity. For example, 0-0.14 M NaCl, which corresponds to physiological salt concentration, can be designated as the “unbound fraction”. The 0.14-0.3 and 0.3-0.6 M NaCl fractions are typically considered “low-affinity” fractions. Typically, the 0.6-1.0 M NaCl fraction, which corresponds to the NaCl concentration range used to dissociate bound FGF7 from HS immobilized on Sepharose, is considered “high affinity”. About 94% of disaccharides will not bind to FGF7 at a physiological concentration of NaCl. Octasaccharide binding can be significantly detected compared to di-, tetra- and hexa-saccharides even in the NaCl range from 0.6-1.0 M. The vast majority, over 99% of total material, doe not bind to FGF7 when 0.6 M NaCl is present. Although yield at 0.6-1.0 M NaCl increases with increasing size of oligomer, the majority is still in the fractions eluted at NaCl concentration below 0.6 M. The yield of the 0.3-0.6 M NaCl fraction increases when the size of oligosaccharide increased along with the concurrent decrease of yield of fractions below 0.3 M NaCl. So, the majority of FGF7-bound HS oligomers appear in the 0.14-0.6 M NaCl fractions that are above the physiological salt concentration but below the concentration required to dissociate FGF7 from immobilized HS. This indicates that the great majority of heparan sulfate chains attached to cell surface proteoglycans may serve as low-affinity storage sites that limit the diffusion of active FGF7 and confine the activity until needed. FGF7 binds crude HS and can be eluted from heparin-Sepharose by about 0.6-1.0 M NaCl. The HS oligomer fraction or part of the fraction eluted from FGF7-affinity matrix by 0.6-1.0 M NaCl represents the biologically relevant and high affinity FGF7- or FGF7/FGFR-interactive species, which is only a very small amount of material from a huge pool of similar composition.

Although specific bioactivities of very low molecular weight HS oligomers is reduced when compared to those of parent crude HS and LMWH, some species in each type of oligomers still retains the desired properties, which resides in a minute amount of the total chains present. The high-activity chains or a portion of high-activity chains exhibiting Factor Xa activity may be enriched by FGF affinity. Octa-, deca- dodeca- and tetradeca-saccharide fractions eluted by 0.6-1.0 M NaCl from FGF7 affinity are enriched in activity from respective crude oligomers. At 0.1 μM concentration, the 0.6-1.0 M NaCl fractions for octa-, deca-, dodeca- and tetradeca-saccharide display 60%, 81%, 82% and 83% inhibition for Factor Xa activity mediated by antithrombin. This is comparable to or even more potent than the 79% inhibition displayed by a synthetic antithrombin (AT)-binding pentasaccharide at the same concentration (0.1 μM) and the 85% inhibition exhibited by crude HS at 0.13 μg/ml. Since the length of these oligomers is longer than a pentasaccharide, the results indicated that the high-affinity FGF7-binding motif is critically related to the antithrombin-binding motif but may still be different in detail. The crude octa-, deca, dodeca- and tetradeca-saccharide at 0.1 μM elicits only 10%, 37%, 66% and 75% inhibition respectively for Factor Xa activity mediated by antithrombin. Fractions eluted by 0-0.14 and 0.14-0.3 M NaCl are essentially devoid of such inhibitory activity. Such activity also increases significantly for the 0.3-0.6 M NaCl fraction when the size of HS oligomer increases, but is still less than that of even crude oligomers.

An aspect of the invention is the use of affinity for FGF and then the activity of the product for assembly of the FGF/HS/FGFR complex as the key element for enrichment of HS oligomers for structural analysis. The activity-guided structural analysis is important to relating structural analysis to the specific bioactivities and dissecting them from the wide range of apparent activities due to the strong electrolyte character of HS.

The present disclosure shows that crude HS octasaccharide has very weak or undetectable activity at 0.3 μM concentration for supporting FGF7 binding to FGFR2IIIb, the only isoform among FGFRs in the epithelial cell membrane context that FGF7 can bind to. Yet the activity of the high affinity fraction, which is less than 1% of the total octasaccharide from crude HS exhibits activity 50% of that elicited by 0.4 μg/ml crude PIMH (about 0.03 μM that gives rise to the peak activity when a 12000-15000 dalton average molecular weight is considered for PIMH) used as the 100% activity standard. The decasaccharide fraction purified by the same method exhibits 120% activity of that of 0.4 μg/ml crude PIMH. Fractions recovered between 0 and 0.14 M NaCl, which represents physiological salt concentration, from all oligomer lengths up to the tetradecasaccharide are devoid of activity of supporting FGF7 binding to FGFR2IIIb. The low-affinity fractions for all the tested oligomer types have very low activity. The high affinity FGF7-bound HS comprises a very small portion of the total pool of crude HS that possesses high activity in support of receptor binding. The low affinity HS, although it constitutes the vast majority, is generally inactive or has very low activity.

The present disclosure demonstrates that (a) FGF7 has the ability to pull down the anticoagulant HS fraction from a vast majority of crude material; (b) the high-affinity FGF7 bound fraction supports maximum activity for FGF7 binding to FGFR, which may be related to the anticoagulant motif; (c) only high-affinity FGF7-binding HS species can elicit physiological activity or triggering FGF-mediated ternary complex formation, while the low-affinity species likely serves as inactive storage sites, protection from degradation, and a reservoir of readily available factor upon environmental perturbation; (d) the motif selected by FGF constitutes only a minute amount of starting material; (e) specificity co-exists with diversity in a pool of HS with heterogeneity in respect to the chain length and composition for interaction with FGF and for FGF signaling; (f) diversity may also exist in specific HS binding motifs among diverse FGFs, FGFRs or the complex FGFR/HS/FGFR, which are of potential utility in biotechnology.

Crude HS, LMWH and size-uniform HS oligomers are very heterogeneous and highly negative-charged entities. In fact, HS is the most acidic and most highly charged macromolecule in biology. Even though each type of HS oligomer is size-selected, it is comprised of length-uniform chains with different chemical composition on a similar basic backbone. The combination of different number and positioning of distinct type of sulfate groups largely makes the heterogeneity possible, which is the basis of its multivalent ability as well as its unique specificity to interact with a variety of distinct proteins. Nevertheless, the tremendous heterogeneity poses difficulty in the isolation, chemical and structural characterization of a specific HS structure that a particular protein interacts with. After separation based on distinct affinity to FGF7, each fraction of HS oligomer remains a heterogeneous mixture, but may have a similar and predominant basic charge/positioning/conformation pattern. In other words, both structural diversity and specificity may still co-exist in the pool of each fraction. For structural characterization of high-affinity FGF7 bound or interactive HS species, further separation based on charge is required.

Anion exchange chromatography analysis of FGF-affinity fractions indicates that with the increase of NaCl concentration, increasing amount of HS oligomer loses the ability to bind to FGF7. The higher the affinity to FGF7 measured by resistance to salt elution, the higher the affinity to the cation matrix with fewer species retained. This again demonstrates selectivity or specificity of FGF7 for specific oligosaccharides out of the crude HS oligosaccharide mixture. The highest affinity HS species for FGF7 with biological activity at the receptor level (of which there is a minute amount in crude HS) has high affinity for the cation matrix. But surprisingly, the converse is not true, i.e., all HS that has high affinity for the cation matrix does not necessarily have high affinity for FGF7. A large part of the highly charged HS appears to be inactive.

It is thought that the affinity for cation matrix is proportional to the degree of sulfation that imparts the charge to the chain. Conventional thinking suggests that HS's activity with regard to promoting the ternary FGF/HS/FGFR complex is a function of the amount of sulfation of the HS, i.e., the binding is simply a function of electrostatics and a greater the degree of sulfation leads to a greater activity of binding. The fact that all highly charged HS does not necessarily exhibit high biological activity suggests that the position of sulfate groups in the chain also contribute. The binding requires greater specificity than previously expected.

Further, mass spectral analysis shows that the highest affinity for FGF7 occurs with an octasaccharide of less sulfation (7 or 8 sulfates) than one with 11 or 12 groups. Thus, the position of 7 or 8 sulfate groups must be critical, and confer the higher affinity to FGF7 in respect to salt elution rather than the total sulfate number. Reduction of the 7,8 sulfated active octamer to disaccharide and subsequent analysis by ion exchange chromatography indicated the presence of predominantly a tri-sulfated disaccharide and ΔHexA2SGlcN6S in the mixture.

This is a clear demonstration of selectivity of FGF7 for HS species based both on factors other than simply charge density, and selectivity for highest biological activity in terms of receptor assembly. These factors are a combination of position and number of sulfates as well as basic disaccharide backbone sequence. FGF7 selects particular types of oligosaccharides from a huge pool of various types of nonspecific oligosaccharides. It is this type of HS that is capable of promoting the ternary FGF/HS/FGFR complex and thereby modulating the FGFR.

One of skill in the art will appreciate that disclosed herein is a method of modulating a FGF receptor in a mammal, comprising providing the animal with substantially purified HS, wherein the substantially purified HS has high affinity for FGF7. According to one embodiment, the mammal is a human. According to one embodiment, the FGF receptor is the FGF2IIIb receptor. The modulation can be either antagonistic or agonistic.

According to a preferred embodiment, the substantially purified HS binds to FGF7 in a medium that is about 0.6 M or greater in NaCl. The HS can be from any viable source of HS. According to one embodiment, the substantially purified HS is a fraction of crude HS. Alternatively, the substantially purified IIS is a fraction of low molecular weight heparin (LMWH). According to an alternative embodiment, the substantially purified HS is synthetic HS.

According to one embodiment of the invention the substantially purified HS has about four to about twenty saccharide units, preferably about eight to about sixteen saccharide units. According to a particularly preferred embodiment, the substantially purified HS has about eight to about twelve saccharide units. According to a particularly preferred embodiment, the substantially purified HS has eight saccharide units and preferably has 7 or 8 sulfates. The substantially purified HS octasaccharide is preferably about 30% sulfonated (if SO₃⁻ is considered, otherwise will be 12% S content in weight). According to a particularly preferred embodiment, the substantially purified HS has a predominant disaccharide composition of a tri-sulfated disaccharide and ΔHexA2SGlcN6S. According to an alternative embodiment, the HS has greater than eight saccharide units, but contains a structural motif that comprises an octasaccharide having 7 or 8 sulfates. Preferably, the structural motif is the high affinity octasaccharide motif described above. According to a particularly preferred embodiment, the motif is an octasaccharide having a predominant disaccharide composition of a tri-sulfated disaccharide and ΔHexA2SGlcN6S. According to an embodiment of the invention, the substantially purified HS has high anticoagulant activity.

According to an aspect of the invention, providing the animal with substantially purified HS oligosaccharides promotes the formation of a complex of the HS with FGF thus prolonging its half-life in the physiological environment. According to another the HS or the FGF-HS complex forms a ternary complex comprising FGF2IIIb receptor, HS, and FGF in the animal. According to one embodiment, providing the animal with substantially purified HS oligosaccharides promotes the formation of a complex of the HS with FGF7. Alternatively, the complex can be of HS with an FGF other than FGF7. Surprisingly, HS with a high affinity for FGF7 is effective for promoting a ternary complex comprising a FGF receptor, HS, and FGF other than FGF7, for example, FGF1 and FGF10.

According to an aspect of the invention, the animal is provided orally with substantially purified HS oligosaccharides. The substantially purified HS can be dispersed in a pharmacologically acceptable liquid or solid carrier. Alternatively, the substantially purified HS can be provided to the animal via topical administration. According to one embodiment, the HS is dispersed in a pharmaceutically acceptable liquid or solid carrier. Examples of such pharmaceutically acceptable formulation include a wound covering selected from the group consisting of a collagen based cream, a collagen based film, a collagen based microcapsule, a collagen based powder, hyaluronic acid, glycosaminoglycans, creams, foams, suture material, and wound dressings.

An embodiment of the present invention comprises providing the animal with substantially purified HS oligosaccharides and with FGF. For example, the animal can be simultaneously provided with substantially purified HS and FGF. The HS and FGF can be dispersed in a pharmaceutically acceptable liquid or solid carrier. According to one embodiment, the FGF is FGF7. Alternatively, FGF(s) other than FGF7, or mixtures of FGF7 and other FGF(s) can be used.

A further aspect of the invention is composition useful for modulating a FGF receptor in a mammal, comprising FGF and substantially purified HS oligosaccharides, wherein the substantially purified HS has high affinity for FGF7. According to one embodiment, the composition comprises FGF7. Alternatively, the composition can comprise an FGF other than FGF7. According to one embodiment, the FGF receptor is FGF2IIIb. According to one embodiment, the substantially purified HS binds to FGF7 in a medium that is about 0.6 M or greater in NaCl. The substantially purified HS preferably has about four to about twenty saccharide units, more preferably about eight to about sixteen saccharide units, and even more preferably about eight to about twelve saccharide units. According to one embodiment, the substantially purified HS is about about 30% sulfonated (if SO₃⁻ is considered, otherwise will be 12% S content in weight). According to a particularly preferred embodiment, the substantially purified HS has eight saccharide units. According to one embodiment the substantially purified HS is an octasaccharide having 7 or 8 sulfates. According to a particularly preferred embodiment, the substantially purified HS has a predominant disaccharide composition of a tri-sulfated disaccharide and ΔHexA2SGlcN6S. According to an alternative embodiment, the HS has greater than eight saccharide units, but contains a structural motif that comprises an octasaccharide having 7 or 8 sulfates. Preferably, the structural motif is the high affinity octasaccharide motif described above. According to a particularly preferred embodiment, the motif is an octasaccharide having a predominant disaccharide composition of a tri-sulfated disaccharide and ΔHexA2SGlcN6S. According to an embodiment of the invention, the substantially purified HS has high anticoagulant activity.

According to one aspect of the invention, providing the animal with substantially purified HS promotes the formation of a ternary complex comprising FGF2IIIb receptor, HS, and FGF7 in the animal.

The composition of the invention can further comprise a pharmaceutically acceptable carrier or diluent.

A still further aspect of the invention is a method for obtaining substantially purified HS oligosaccharides that modulates a FGF receptor in a mammal, the method comprising: obtaining an affinity matrix comprising a fibroblast growth factor that preferentially binds to HS that modulates the a FGF receptor in a mammal, contacting the affinity matrix with a mixture comprising HS, separating the non-bound material from the bound material, and obtaining substantially purified HS as the bound material. Preferably, the FGF receptor is FGFR2IIIb and the fibroblast growth factor is FGF7. Preferably, the substantially purified HS oligosaccharides binds the affinity matrix in a solution that is greater than about 0.6 M in NaCl.

Generally, the mixture comprising HS can be any mixture comprising HS. The mixture comprising HS oligosaccharides is from crude heparin, low molecular weight heparin, or any of their derivatives.

According to one embodiment of the invention, the method further comprises contacting the crude heparin with a heparinase before contacting the affinity matrix with the crude heparin.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### HS with Highest Affinity for FGF7 and Highest Anticoagulant Activity Exhibits Highest Activity for FGF7-HS-FGFR2IIIb Complex Formation

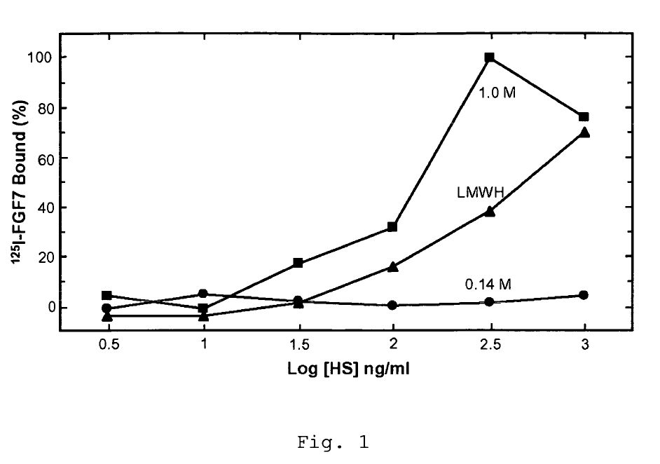

It has been shown that scalable bacterial-derived quantities of specifically FGF7 relative to FGF1 and FGF2 selectively captures anticoagulant HS from diverse crude HS preparations if precautions are taken in quality of preparation of recombinant material and immobilization strategies for affinity chromatography (Ye, S., Luo, Y., Lu, W., Jones, R. B., Linhardt, R. J., Capila, I., Toida, T., Kan, M., Pelletier, H., and McKeehan, W. L. Structural basis for interaction of FGF-1, FGF-2, and FGF-7 with different heparan sulfate motifs. Biochemistry (2001); Luo, Y., H-H Cho, R. B. Jones, and W. L. McKeehan, Improved Yield of Recombinant Fibroblast Growth Factor 7 (FGF7/KGF) from Bacteria in High Magnesium Chloride. Protein Expression & Purification (2003); Luo, Y., H—H, Cho and W. L. McKeehan. Biospecific extraction and Neutralization of anticoagulant heparin with fibroblast growth factors (FGF). J. Pharmaceutical Science and in U.S. Pat. No. 6,812,221). The specific anticoagulant activity increased proportional to the affinity to FGF7 measured by resistance to elution with graded concentrations of NaCl with the highest activity eluting between 0.60 and 1.0 M (Luo, Y., H—H, Cho and W. L. McKeehan. Biospecific extraction and Neutralization of anticoagulant heparin with fibroblast growth factors (FGF). J. Pharmaceutical Science). A FGFR binding assay, which was done with 4 ng/ml (10⁶ cpm) ¹²⁵I-FGF7 and FGFR2IIIb-GST expressed and anchored on the surface of Sf-9 cells or FGFR2IIIb-GST immobilized on the GSH-Sepharose after purified from Sf-9 cells, in PBS buffer containing 1 mg/ml BSA, 0.15 M NaCl, 10 mM MgCl₂ and 0.1 mM DTT was performed. The oligosaccharide fractions were added either together with ¹²⁵I-FGF7, or added alone to the receptor and then washed with buffer to eliminate the nonspecific oligosaccharide binding prior to the addition of ¹²⁵I-FGF7. The maximal binding radioactivity that can be reached was considered as 100%. FIG. 1 shows that the corresponding fraction with highest affinity for FGF7 measured by salt resistance and highest anticoagulant activity from commercial low molecular weight heparin (LMWH) also exhibited highest activity in support of ¹²⁵I-FGF7 binding to insect cells expressing membrane anchored FGFR2IIIb-GST. Concentrations of HS required to support half-maximal binding of ¹²⁵I-FGF7 was about 450 and 140 ng/ml for crude LMWH, fractions eluted between 0.6-1.0 M NaCl, respectively. Activity of the unbound fraction at 0.14 M NaCl and the small amount of material that eluted above 1.0 M NaCl (not shown) was negligible. A correlation between affinity of HS for FGF7, increase in anticoagulation activity and ability to form binary complexes of HS-FGFR2IIIb by preincubation with cell free FGFR2IIIb-GST prior to introduction of FGF7 was verified by covalent affinity crosslinking of FGF7 to FGFR2IIIb in separate experiments (not shown). These results confirm that anticoagulant HS that exhibits FGFR-independent high affinity binding to FGF7 participates in both the independent interaction of HS with FGFR2IIIb and formation of the ternary FGF7-HS-FGFR2IIIb complex. High affinity binding to FGF7 is a feasible route to enrichment of oligosaccharides of distinct structure that are involved in formation of the ternary FGFR signaling complex in quantities sufficient for structural characterization.

### Example 2

### Controlled Production and Size-Selection of HS Oligosaccharides and Properties of Short Size-Selected Sulfated Oligomers

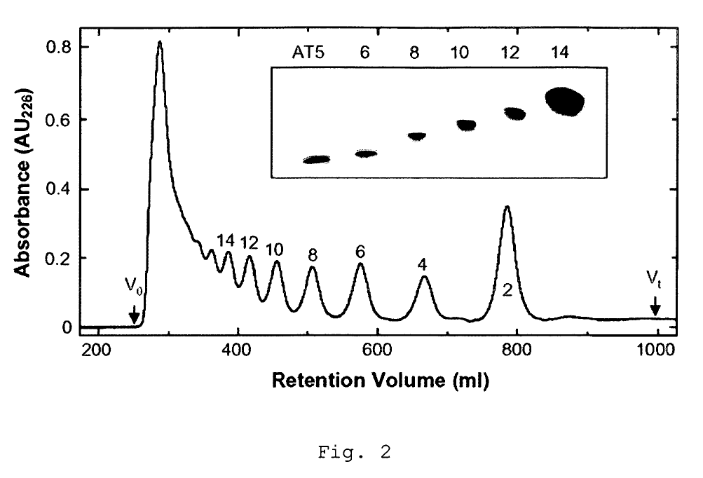

Heparinase 1, was used to cleave a crude porcine intestinal mucosal heparin (PIMH) mixture with chain length range from about 12 to 60 disaccharides. Since heparinase 1 recognizes and cleaves the anticoagulant motif and presumably FGF/FGFR-specific motifs, a 10 to 30% cleavage rate was found to be the best compromise between preservation of anticoagulant activity, ability to promote FGF7 binding of the total spectrum of oligosaccharides of defined size and total yields ranging from a disaccharide to tetradecasaccharide. 1.0 gram heparin from porcine intestinal mucosa (6000-30000 Da, 170 USP units/mg, Sigma, St. Louis, Mo.) was dissolved in 10 ml buffer of 100 mM Sodium Acetate, 2.5 mM Calcium Acetate, pH 7.4, and 1 mM DTT, and filtered through 0.22 μm Tuffryn Membranes (Pall Corporation, Ann Arbor, Mich.). Then 40 Sigma units of heparinase 1 (Sigma, St. Louis, Mo.) was added, and the mixture was incubated for about 18 hrs at 37° C. to achieve a 10-30% partial digest as compared to exhaustive digest in which absorbance reaches to maximum and did not increase anymore with new addition of enzymes, by monitoring the increase of product absorbance at 226 nm because of the formation of double bond between the C4 and C5 atom on the non-reducing end after the β-eliminative endolytic cleavage. The heparinase 1 was inactivated by incubation at 75° C. for 5 min. 300 mg resulting mixture was passed through a column of porous polyacrylamide-based polymer matrix (2.6×190 cm) with separation range of 1500-20000 dalton, at a flow rate of 0.3 ml per minute in a heat-decomposable buffer. The column is capable of separating oligomers with maximum size of hexadecasaccharide at maximum sample load (FIG. 2). Although peak overlapping occurs at dodecasaccharide, by controlling the sample loading, we can control the separation resolution and efficiency, thus the final purity of respective HS oligomers can be sufficed. Each peak corresponding to individual HS oligosaccharide was then collected, heated at 70° C. for 1 hour, and lyophilized. The dried and slightly yellowish powder of each peak was re-dissolved in water, and fractionated for one more time to insure size homogeneity. After second purification, the lyophilisate was desalted on a 5 ml Sephadex G-25 column (Amersham Pharmacia Biotech, Piscataway, N.J.), and concentrated by lyophilization. The quantity of each oligosaccharide was determined first by 1,9-dimethy-methylene blue (DMB) (Biocolor Ltd., Newtownabbey, Northern Ireland) and then by modified H₂SO₄-borate-carbazol assay.

Because of the variation of retention volume of Bio-Gel P-10 column over time, the purity and size of oligosaccharides were further confirmed by 16-36% gradient PAGE. The 16-36% gradient gel was formed in a Model 485 Gradient Former (Bio-Rad, Hercules, Calif.). The oligosaccharide sample was in a solution of 50% sucrose, 0.04% bromophenol blue and 0.4% phenol red, and run through the gel with an upper chamber buffer of 0.2 M Tris and 1.24 M Glycine, and a lower chamber buffer of 0.1 M Boric acid, 0.1 M Tris-HCl pH 8.3 and 0.01 M EDTA, at 400 volts for 4 hours. Oligosaccharides in gel were visualized by Alcian Blue staining and then the background was destained by 5% acetic acid (FIG. 2, inset). If band crossover occurred, the oligosaccharide was purified at least one more time by the Bio-Gel P-10 column. There are not overlapping bands that can be seen between two adjacent oligomers with a mass difference of a disaccharide (FIG. 2, inset). The purity is the basis of the further study of structure-activity relationships (SAR).

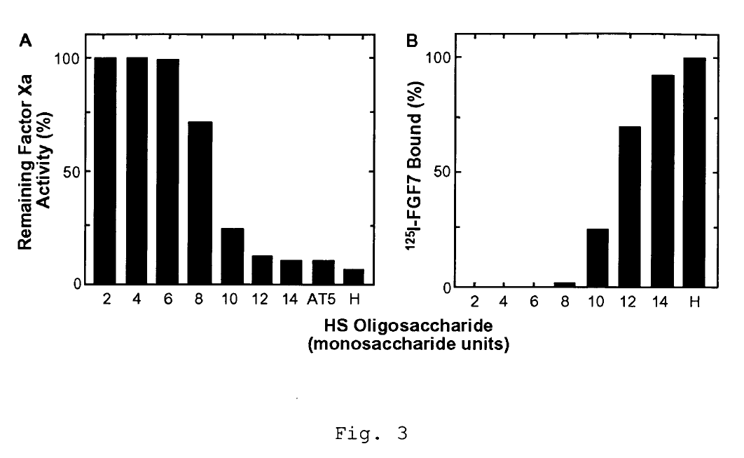

Reduction of size to short oligomers greatly reduced the ability of HS to accelerate antithrombin-mediated anti-Factor Xa, activity and to support FGF7 binding to FGF receptor (FGFR) (FIG. 3). Disaccharides through tetradecasaccharides were separated by gel filtration (FIG. 2) and subsequently examined for inhibition of Factor Xa activity and promotion of FGF7 binding to FGFR2IIIb prior to further purification. At 0.3 μM concentration, the anticoagulant activity of disaccharide, tetrasaccharide and hexasaccharide mixtures was undetectable while octasaccharide and decasaccharide reduced Factor Xa activity to 70% and 25% of controls, respectively (FIG. 3A). Activity of the dodecasaccharide and tetradecasaccharide mixtures was near the 90% inhibition exhibited by 0.3 μM of a synthetic antithrombin-binding pentasaccharide and 0.4 μg per ml of the unsized porcine intestinal mucous heparin (H) that was the maximum inhibition observed under the assay conditions. The ability of the crude mixtures of the oligosaccharides of defined size to support FGF7 binding to FGFR2IIIb was compared. FGFR2IIIb binding was expressed as a percent of that supported by 0.4 μg/ml crude PIMH that supported maximum binding under the assay conditions (100% binding values). At 0.3 μM, the disaccharide, tetrasaccharide and hexasaccharide mixture failed to support detectable FGF7 binding (FIG. 3B). At the same level, octa-, deca-, dodeca- and tetradecasaccharides exhibited 2%, 24%, 70% and 90% of the standard 0.4 μg/ml crude PIMH. These data indicate that both the binding of FGF7 to FGFR2IIIb and anticoagulant activity increases with increasing lengths of oligosaccharide mixtures. An octasaccharide mixture is the shortest with which both anticoagulant activity and FGF7 binding can be detected at all by this method of preparation and assay. Maximal binding activity lags maximal anticoagulant activity by one disaccharide unit.

### Example 3

### Isolation of FGF7-Bound HS Oligomers with Distinct Affinity

High quality GST-FGF7 and FGF7 was produced in the presence of 30 mM MgCl₂ in BL21 DE3 pLysS bacteria with significantly improved yield. GST-FGF7 was purified first by batch Heparin-Sepharose chromatography or GSH-Sepharose chromatography, then Heparin-Sepharose chromatography in FPLC System with linear gradient of Sodium Chloride (NaCl) in buffer A of 10 mM Tris-HCl, pH7.4, 0.1 mM DTT and 0.02% NaN₃. A FGF7 affinity matrix was then made by immobilizing GST-FGF7 on GSH-Sepharose column by bioaffinity between GST and GSH partnership, or on NHS-activated Agarose matrix by covalent bond formation. Both immobilization methods preserved the intact heparin-binding ability of GST-FGF7 and FGF7 (data not shown). According to the latter method, 20-30 mg pure GST-FGF7 or FGF7 was desalted and buffer-exchanged in a buffer of 0.2 M Sodium Hydrogencarbonate, 0.3 M NaCl, pH7.8, and complexed with N-acetylheparin (Sigma, St. Louis, Mo.) and concentrated in Centricon Plus-20 (MWCO 10000 Da, Millipore, Bedford, Mass.). The GST-FGF7/N-acetyl-heparin complex was immobilized covalently through primary amino groups of GST-FGF7 or FGF7 onto the pre-packed NHS-activated Agarose column (Amersham Pharmacia Biotech, Piscataway, N.J.), and the excessive reactive groups in column were blocked by 0.2 M ethanolamine, pH8.0. The resulting GST-FGF7 column was washed with linear gradients of 0.14 to 1.3 M, then 1.3 to 0.14 M of NaCl in buffer A at 1 ml/min, and equilibrated in buffer A containing 0.14 M NaCl. Since HS octasaccharide was the threshold lower limit in size that began to elicit activities for both inhibiting Factor Xa and supporting FGF7 binding, HS oligomers from octa- to tetradeca-saccharide were chosen for the studies of affinity-purification and subsequent SAR. NaCl at different concentrations was used to differentiate bound oligomers from FGF7 affinity. The 0-0.14 M NaCl fraction was designated as the “unbound fraction,” which corresponds to physiological salt concentration. The 0.14-0.3 and 0.3-0.6 M NaCl fractions were designated “low-affinity” fractions. Elution between 0.60 and 1.0 M salt was defined as the “high-affinity” fraction since FGF7 is retained on Heparin-Sepharose column at salt concentrations up to 0.60 M and elutes at 1.0 M. Each pool of first-time extract for 0-0.14, 0.14-0.3, 0.3-0.6 and 0.6-1.0 M NaCl fractions was re-extracted using another newly prepared affinity column. The resulting pools were boiled, filtered, lyophilized, and then desalted by Sephadex G-25. The binding of disaccharide and tetrasaccharide was undetectable when passed through the FGF7 affinity column in the presence of NaCl above 0.6 M. Binding of the fraction in the range of 0.3-0.6 M NaCl is less than 10% (Table 1). About 94% of the disaccharides will not bind to FGF7 at a physiological concentration of NaCl. The amount of oligosaccharide bound with high affinity increased progressively with the hexasaccharide (0.2%) through the tetradecasaccharide (4%). Notably the largest increase in yield of oligosaccharides with high affinity for FGF7 that was also evident in the 0.60 M elution occurs with an increase from 6 to 8 units. The results suggest that only a very small fraction of HS oligosaccharide mixtures with a minimum size of 6 to 8 units have high affinity for FGF7 while the majority binds with a low affinity eluting below 0.6 M NaCl, indicating that the oligosaccharides with high affinity in each sized mixture are rare.

### Example 4

### Anticoagulant and FGF7 to FGFR2IIIb Binding Activity of FGF7-Affinity Purified Oligosaccharides

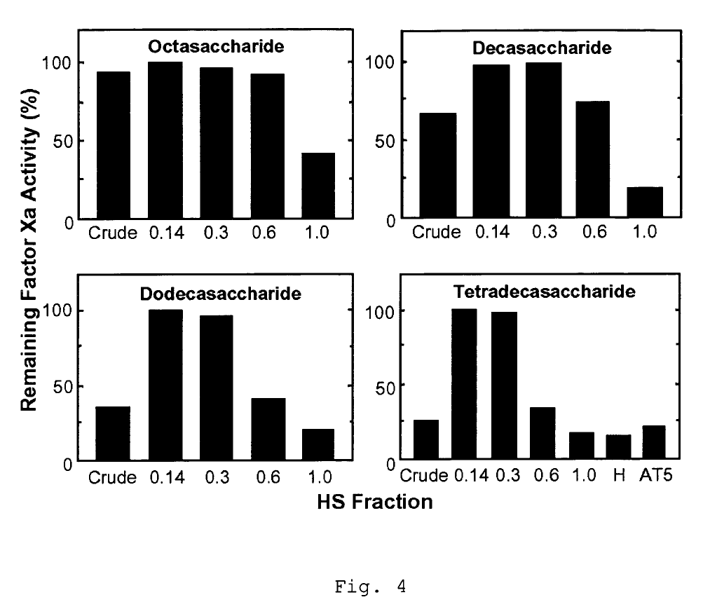

The anticoagulant activity of the oligosaccharides with graded affinity for FGF7 based on salt resistance in the antithrombin-mediated inhibition of Factor Xa assay was determined. 10 μl of sample containing a defined amount of oligosaccharides of different fractions from FGF7 affinity column was mixed with 10 μl of 10 μg per ml solution of antithrombin (Sigma, St. Louis, Mo.) in 20 mM Tris-HCl (pH 7.4), 0.15 M NaCl and 10 mM CaCl₂. 70 μl of 200 ng/ml Factor Xa (New England Biolabs, Beverly, Mass.) was added and incubated at 37° C. for 3 min. The mixture was then incubated with 10 μl of 2.33 mg/ml chromogenic substrate Chromozym X (Roche Molecular Biochemicals, Indianapolis, Ind.) at 37° C. for 4 min. The reaction was stopped by 10 μl glacial acetic acid. The residual Factor Xa activity was determined at 405 nm. The Factor Xa activity in the presence of antithrombin, but absence of heparin oligosaccharides was defined as 100 percent activity. Similar to the entire hexasaccharide mixture, no activity could be demonstrated in the small amount of material that bound to FGF7 at highest affinity (0.60-1.0 M salt). At 0.1 μM, the octasaccharide that bound with highest affinity (0.60-1.0 M salt) reduced Factor Xa activity by 60% while very little activity was observed in the 0.60 M eluate representing the fraction with moderate affinity for FGF7 (FIG. 4). The high affinity-binding fraction of the decasaccharide mixture and longer oligosaccharides exhibited maximal inhibition of Factor Xa in the assay. However, crude octa-, deca-, dodeca-, and tetradeca-saccharide elicit 10%, 37%, 66% and 75% anticoagulant activity, respectively, at 0.1 μM. Unbound fractions and 0.3 M NaCl fractions are essentially devoid of such activity (FIG. 4). Such activity also increases significantly for the 0.6 M NaCl fraction when the size of oligosaccharides increases, but is still less than that of high-affinity oligosaccharides This confirms that oligosaccharides with highest affinity for FGF7 exhibit the highest anticoagulant activity and are thus potentially the best candidates for formation of a specific ternary FGF7-HS-FGFR2IIIb complex.

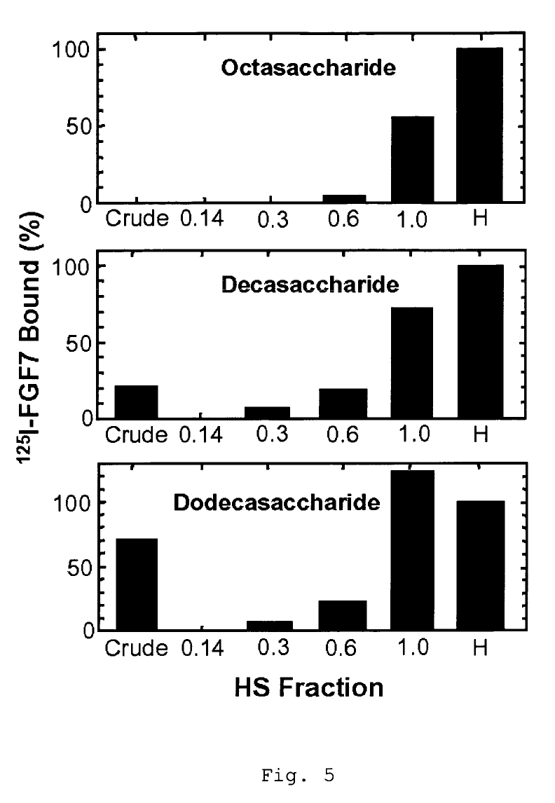

Fractions from the FGF7-affinity column were then screened for support of binding of FGF7 to FGFR2IIIb as described in Example 1. Independent of length oliogosaccharides that failed to bind FGF7 at physiological salt failed to support binding to FGFR2IIIb. At 0.3 μM no activity could be detected in the small amount of hexasaccharide that was retained above 0.60 M salt. Although 0.3 μM of crude octasaccharide was at the threshold for detection of binding activity (FIG. 3), the same amount of high affinity FGF7-bound octasaccharide that is less than 1 percent of the crude oligosaccharide mixture exhibited about 50 percent the activity of the PIMH standard (FIG. 5). Activity in the lower affinity fraction eluting at 0.60 M salt was barely detectable. Similar to the unfractionated oligosaccharide starting material, activity of the high affinity FGF7-bound oligosaccharides increased with increasing length. The high affinity decasaccharide and dodecasaccharide exhibited 70 and 120 percent, respectively, of the PIMH standard. These results demonstrate that rare anticoagulant octasaccharides with highest affinity for FGF7 as short as 8 monosaccharides in length supports formation of a specific complex of FGF7 and FGFR2IIIb, indicating that rare specific motif in HS chains plays a critical role in dictating the biological activity of FGF7. In contrast, the LA oligosaccharides from FGF7 that constitute the majority of the oligosaccharide preparations had no or little activity for the ternary complex formation. These fractions probably represent the broad spectrum of HS motifs that play an FGFR-independent role in storage, stability and trafficking of FGF7 in the extracellular environment.

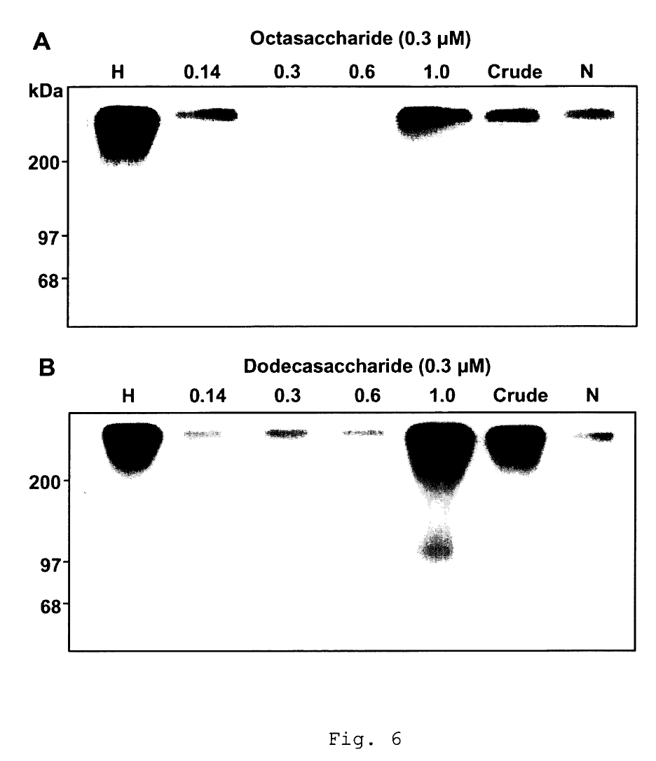

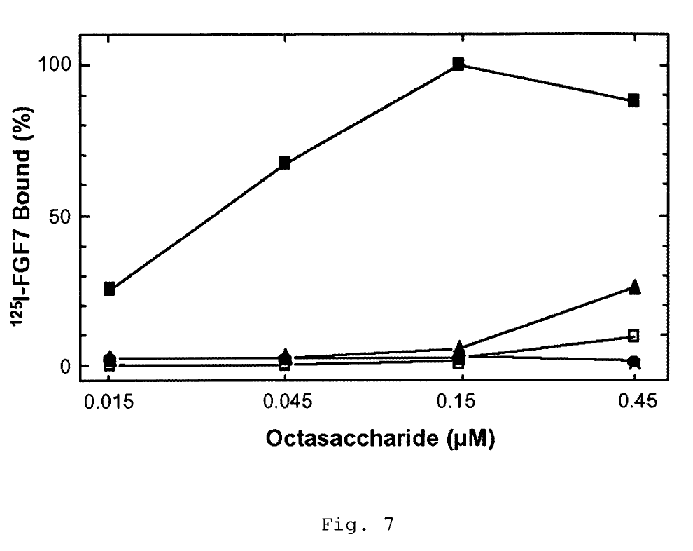

Covalent affinity crosslinking analysis confirmed that the FGF7 binding supported by the purified oligosaccharide fraction reflected authentic high affinity binding to FGFR2IIIb that was crosslinkable by amine-reactive DSS with a 11.4 Å spacer arm (FIG. 6). To estimate efficacy of the FGF7-affinity purification of the high affinity active octasaccharide relative to the crude octasaccharide mixture that exhibited little or no activity at 0.3 μM, the dose-dependent activity of purified octasaccharide to the crude fraction was compared (FIG. 7). A detection limit of purified octasaccharide was about 1 nM with a half of maximum and maximum activity at 30 nM and 0.15 μM, respectively. Crude octasaccharide mixture could not achieve half-maximal activity due to the heterogeneity that elicits a complex effect of activation, inhibition and interference. The efficacy of FGF7-affinity for purification of an active octasaccharide is thus remarkable. This high level of purification suggests that the active motif that can be restricted to an octasaccharide that is required for specific FGF7-FGFR2IIIb complex formation may be rare and therefore structurally specific.

### Example 5

### Charge-Based Separation of Fractions of HS Oligomers

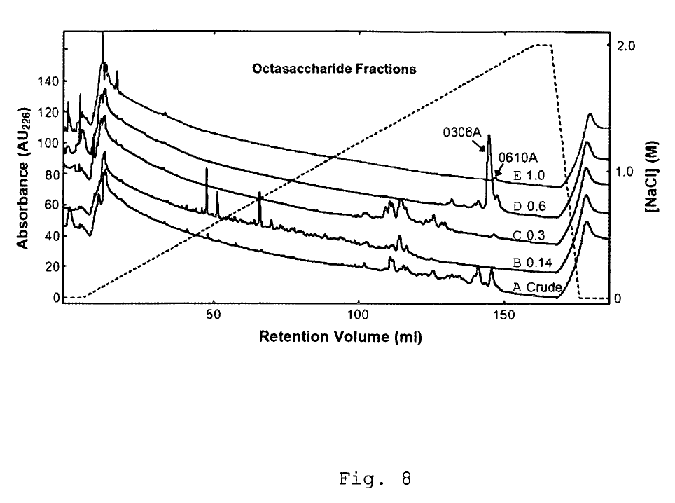

To determine the extent of heterogeneity of the FGF7-affinity fractionated octasaccharides, the fractions eluted from the FGF7-affinity columns were first subjected to analysis by strong anion exchange chromatography (FIG. 8). Each fraction of octasaccharide from GST-FGF7 or FGF7 column was further resolved according to their differential charge pattern by anion exchange on a Propac PA1 column (4 mm×250 mm) (Dionex, Sunnyvale, Calif.) eluted with a linear gradient of NaCl from 0 (pump A1) to 2.0 M (pump B1) in H₂O—HCl (18.2 megohms/cm at 25° C., pH 3-3.5) over a period of 160 min at 1 ml/min on AKTApurifier HPLC monitored at 226 nm. Oligosaccharide fractions corresponding to individual peak was pooled, desalted and dried as described above, and re-dissolved in H₂O. Crude octasaccharide starting material applied at 100 μg displayed a number of peaks dispersed along a broad range of NaCl concentration, which demonstrated the heterogeneity of HS in fractions with chain length as short as 8 monosaccharides (FIG. 8A). The unbound fraction at 0-0.14 M also exhibited dispersed peaks that eluted from the anion exchange column but major peaks occurred at about 0.45-0.75 M NaCl as well as 1.6 M NaCl (FIG. 8B). It is interesting that among the FGF7-unbound HS species under physiological salt concentration highly charged species also exist, which implies that highly charged HS might not necessarily exhibit high affinity to FGF7. The 0.14-0.3 M NaCl fraction revealed peaks concentrated between 1.2-1.6 M NaCl (FIG. 8C). In contrast, the 0.3-0.6 M NaCl fraction showed one predominant peak at 1.8 M NaCl (named 0306A) with some minor peaks (FIG. 8D). Due to the scarcity of material in the 0.6-1.0 M NaCl fraction, 10 μg was loaded onto the anion exchange column, and the chromatogram shown a single peak (named 0610A) at about 1.8 M NaCl (FIG. 8E), while heterogeneity increased as the affinity in respect to salt elution decreased, indicating that the high-affinity octasaccharides contains rare motif.

### Example 6

### Structural Analysis of FGF7 Affinity Purified Oligosaccharides with Ability to Assemble the FGFR Ternary Complex

The major peaks within the 0.3-0.6 and 0.6-1.0 M elutions from FGF7 affinity and subsequent anion-exchange chromatography, 0306A and 0610A (FIGS. 8D, 8E arrow), were analyzed by MALDI-TOF mass spectrometry. To protect the labile structure of sugar chain and functional groups from destruction by the high energy beam required for analysis and to enhance the output of signal, a complex with basic peptide (Arg-Gly)₁₉-Arg was applied in the analysis. About 11 ng of high affinity octasaccharide (peak 0610A from FIG. 8) (A) or octasaccharide with lower affinity (peak 0306A) (B) (2 μl) for FGF7 was mixed with about 50 ng synthetic peptide carrier [(Arg-Gly)₁₉-Arg] (1 μl) in the presence of 4 μl 15 mg/ml caffeic acid in 40% aqueous acetonitrile. Aliquots (2 μl) of the mixture were deposited on a polished stainless steel chip, dried, and analyzed in a Bruker Autoflex MALDI-TOF mass spectrometer in a linear positive mode with 120 ns delayed extraction and 2000 Da mass gate. Observed in each mass spectrum were the (M+H)⁺ ions of the basic peptide and the (M+H)⁺ ions of a 1:1 peptide/saccharide complex.

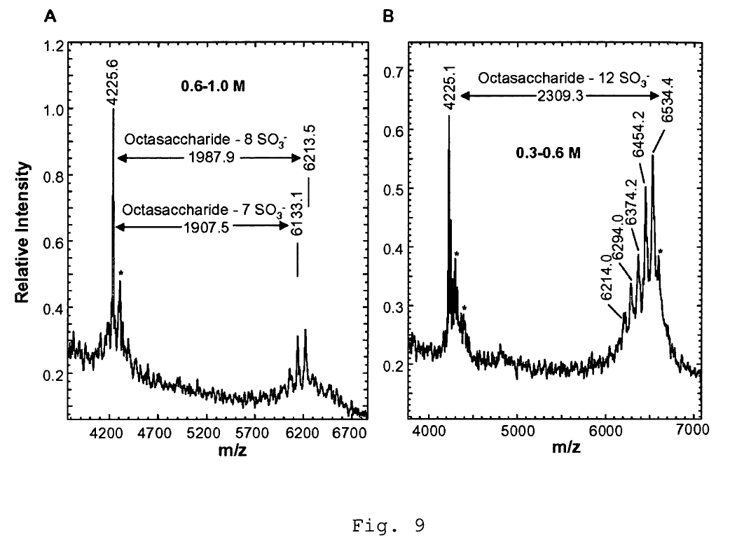

Typically, a 1:1 molar complex was observed between octasaccharide and the peptide. A theoretical mass of the [complex+H]⁺ can be predicted by the formula [m/z=4225.61+337.29N+80.06N_{SO3}+42.04N_{Oac}] given N, N_{S03} and N_{OAC} are all positive integers. The recorded m/z value (corresponding to the m value for a single charge) of saccharide represents the difference in recorded mass between the peptide-saccharide complex and the peptide alone (4225.61). The 0610A peak gave two predominant signals at experimental m/z of 1908.47 and 1987.9 with the latter signal stronger than the former. These molecular weights corresponded to an octasaccharide with 7 and 8 sulfates, respectively (FIG. 9A). Surprisingly, the 0306A peak that elutes at lower salt on ion exchange and have low anticoagulant activity and failed to support the FHR complex formation, gave about 5 peaks on mass spectra, two major peaks at 2229.0 and 2309.28, and three minor peaks at 2149.12, 2069.85 and 1988.91. The two major peaks corresponded to octasaccharide with 11 and 12 sulfates, respectively (FIG. 9B). The results indicate that as few as two species of octamer binds FGF7 with highest affinity (elution at 1.0 M salt), exhibits highest activity for FHR complex formation, and highest anticoagulant activity. These results show that these activities are not simply related to charge density and suggest a high degree of structural specificity of the motif within the octamer in respect to monosaccharide sequence and sulfation pattern.

### Example 7

### Disaccharide Composition of High-Affinity and High-Activity Octasaccharides

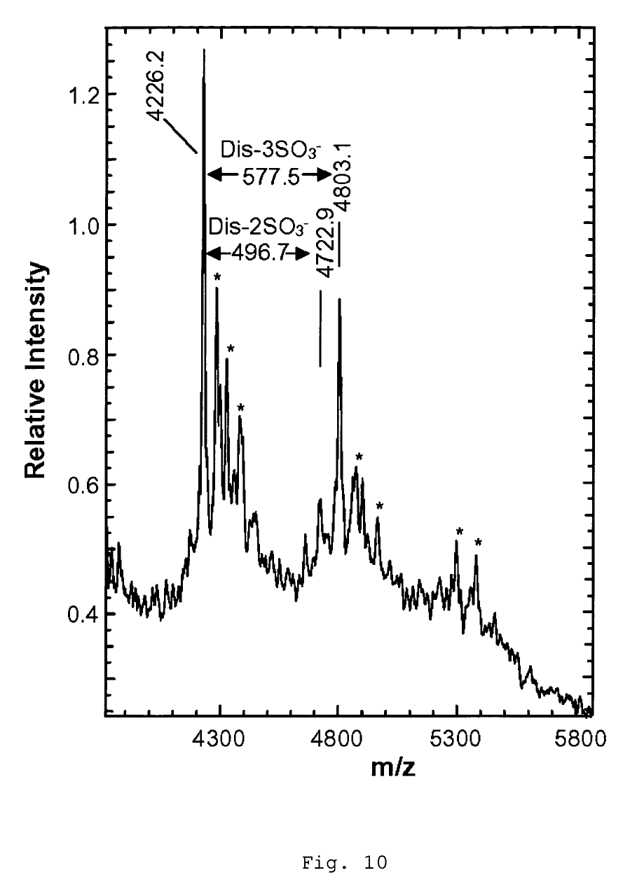

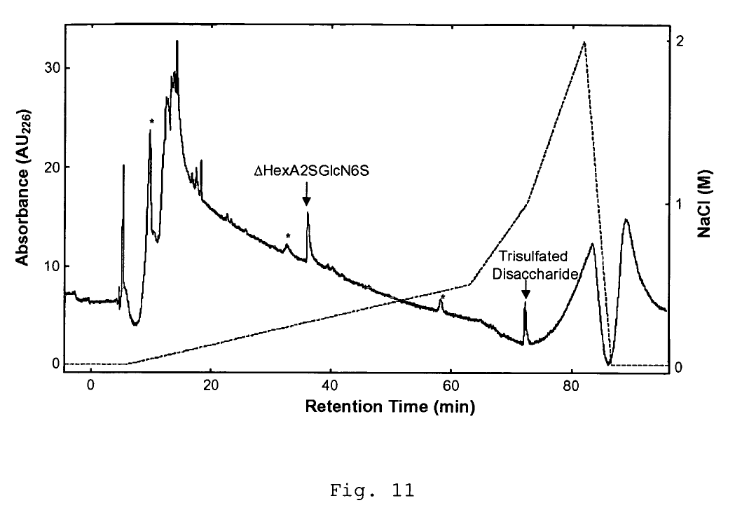

Disaccharide compositional analysis of the fractions was conducted as follows: About 4 μg of the 0.6-1.0 M NaCl fraction of octasaccharides was exhaustively digested by a combination of heparinase 1, 2 and 3 overnight at 37° C. The digestion mixture was desalted by Sephadex G-25 in water and concentrated by centrifugal evaporation. MALDI-TOF mass spectrometry analysis of the digestion mixture as described above yielded two signals with m/z value of 577.46 and 496.71, which corresponded to disaccharides with 3 and 2 sulfates respectively (FIG. 10). The digestion mixture was also subjected to both anion exchange and ion-pair reverse phase chromatography. The anion exchange chromatography was done as described above except for variation in the elution gradient (FIG. 11), which was gradient elution to 25% B1 over 60 min, gradient elution to 50% B1 over 10 min, followed by to 100% B1 over 10 min all at a flow rate of 1 ml/min. The column was then washed with gradient from 100% B1 to 0.2% B1 over 5 min and re-equilibrated with 0.2% B1 for 10 min. The result indicate the presence of predominantly a disaccharide bearing three sulfates at about 0.95 M NaCl and a disaccharide ΔHexA2SGlcN6S at about 0.3 M NaCl.

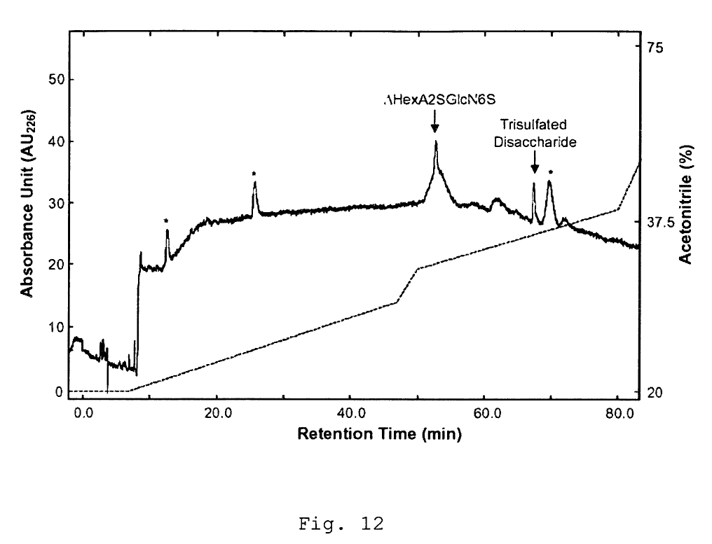

The ion-pair reverse phase chromatography was conducted on a Supelcosil LC-18 column (4.6 mm×250 mm) (Supelco, Bellefonte, Pa.) connected to a Spheri-5 RP-18 precolumn (4.6 mm×30 mm) housed in 3 cm MPLC holder (Brownlee Labs). A gradient elution was performed using a binary mobile phase system composed of 20% (v/v) aqueous acetonitrile (pump A2) and 75% (v/v) aqueous acetonitrile (pump B2). 0.01 M Tetrabutylammonium hydroxide (in 40% stock solution) was added to both A2 and B2, and adjusted pH 6.7 by phosphoric acid. The multi-step gradient scheme for mobile phase was established by pilot study with heparin disaccharide standards, which was isocratic elution with 100% A2 for 7 min, gradient elution to 27% B2 over 40 min, faster gradient to 37% B2 over 3 min, gradient to 55% over 30 min, followed by gradient to 100% B2 over 10 min at a flow-rate of 1.2 ml/min. The column was returned to 100% A2 over 5 min, and then continued for 10 minutes. The column eluent was monitored by absorbance at 226 nm. The result also confirmed the presence of predominantly the two disaccharides at about 47% and 42% aqueous acetonitrile (FIG. 12).

### Example 8

### Properties of FGF1-Bound Oligosaccharides

Compared to FGF7, which has a unique structure in the HS-binding domain and unique specificity for rare HS motif and FGFR2IIIb, FGF1 exhibits a highly charged composition in the HS-binding domain, and a spectrum of activity for all the FGFRs. The difference in the structure of HS-binding domain predicts different requirements for HS motifs. The same procedures as used in isolating and analyzing the binding oligosaccharides for FGF7 were applied to FGF1. The 0-0.14, 0.14-0.3, 0.3-0.6, 0.6-1.0 and 1.0-1.7 M NaCl schemes were used to distinguish different FGF-1 bound oligosaccharide fractions. The 0.6-1.0 and 1.0-1.7 M NaCl fractions were defined as high affinity for FGF1. Results showed that FGF1 also requires minimal 6-8 monosaccharides for high affinity binding, but binds more oligosaccharides with both low affinity and high affinity. There is at least 6-fold more binding of octasaccharides with high affinity to FGF1 than to FGF7 (Table 2).

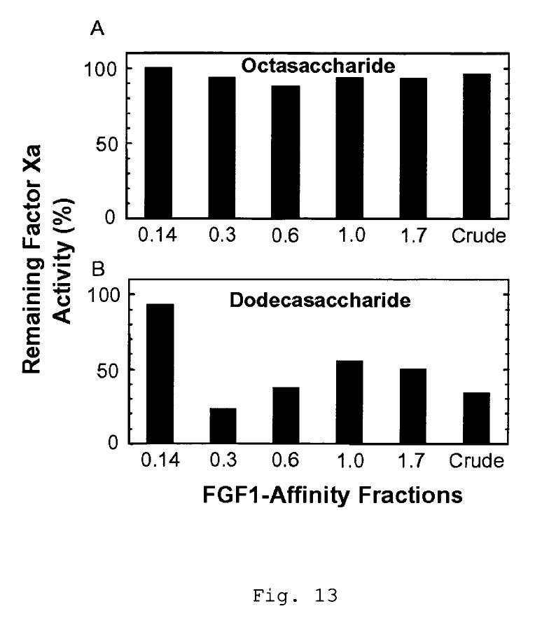

Each fraction of crude octasaccharides and dodecasaccharides from FGF1 affinity was assayed for activity of inhibition of Factor Xa. All the fractions of octasaccharides exhibited similar negligible activity as the crude octasaccharides at 0.1 μM. Crude dodecasaccharides had 65% inhibition for Factor Xa activity mediated by antithrombin (FIG. 13A). The 0.14-0.3, 0.3-0.6, 0.6-1.0 and 1.0-1.7 M NaCl fractions of dodecasaccharides displayed 76%, 62%, 43% and 50% inhibition for Factor Xa activity respectively. The unbound fraction was essentially devoid of such activity (FIG. 13B). The results suggest that unlike FGF7, FGF1 cannot enrich the inhibition activity for Factor Xa with high affinity, indicating that FGF1 may not necessarily require anticoagulant motif for binding HS with high affinity, although the presence of anticoagulant motif may not reject FGF1 binding.

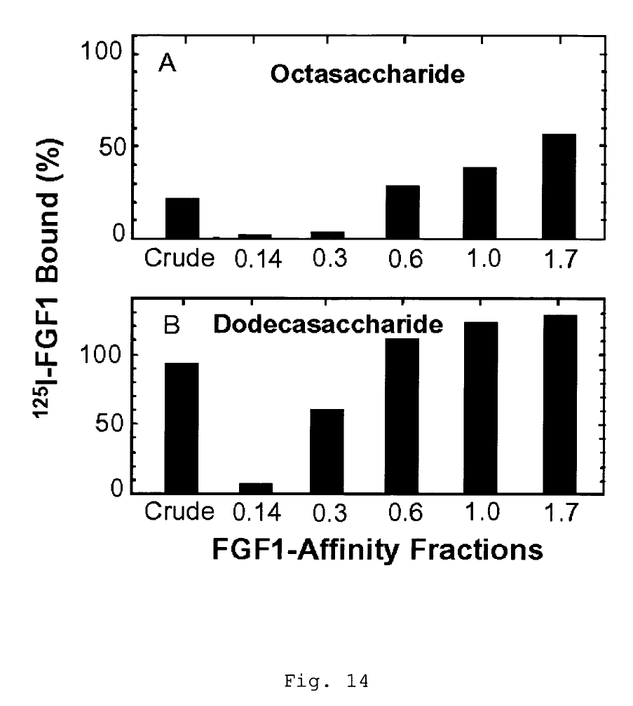

Fractions from the FGF1-affinity column were also screened for support of binding of FGF1 to FGFR1IIIc. At 0.3 μM crude octasaccharides and dodecasaccharides displayed 23% and 94% the activity of the PIMH at 0.4 μg/ml (FIG. 14). Although high-affinity fractions of 0.6-1.0 and 1.0-1.7 M NaCl for octasaccharides and dodecasaccharides elicited highest activity, which were 39% and 55%, and 123% and 129% respectively, for support of FGF1 binding to FGFR1IIIc, unlike that of FGF7, the 0.3-0.6 M NaCl fraction still remains significant activity (FIG. 14A, 14B). The results indicate that FGF1 interacts with broader classes of HS motifs with high affinity and less specificity for support the binding to FGFR.

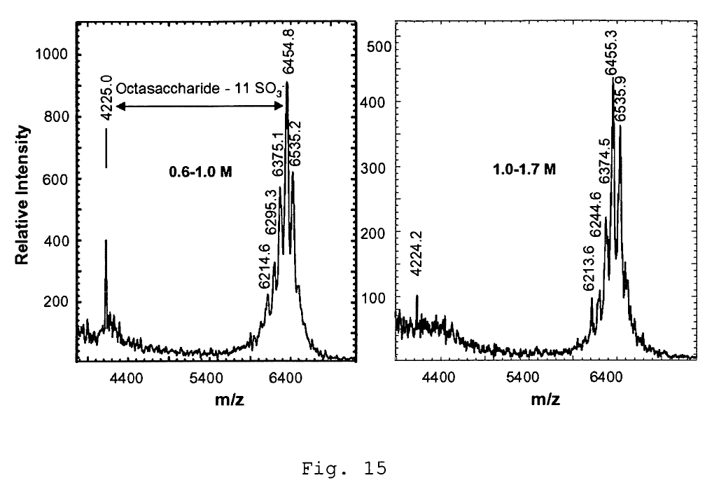

Fractions of the octasaccharides eluting from FGF1 affinity matrices at 0.6-1.0 and 1.0-1.7 M NaCl were analyzed by MALDI-TOF-MS (FIG. 15A, 15B). Both fractions exhibited a spectrum of octasaccharide with predominant high charge species of 10-12 sulfates. The results indicate the FGF1 interacts with a broader spectrum of oligosaccharide motifs related to high charge density, which have less or no structural specificity.

TABLE 1
Affinity of oligosaccharides of defined length for FGF7

Percent of applied oligosaccharide

| HS oligosaccharide | NaCl (M) | | | |
|---|---|---|---|---|
| (Monosaccharide units) | 0.14 | 0.3 | 0.6 | 1.0 |
| 2 | 94 | 5.8 | 0.2 | 0 |
| 4 | 56.3 | 35.6 | 8.1 | 0 |
| 6 | 32.4 | 60.1 | 7.2 | 0.2 |
| 8 | 29.4 | 30.4 | 39.1 | 0.7 |
| 10 | 27.2 | 25.4 | 45.2 | 1.2 |
| 12 | 22.8 | 34.6 | 38.4 | 2.5 |
| 14 | 27.8 | 25.6 | 40.6 | 3.7 |

TABLE 2
Affinity of oligosaccharides of defined length for FGF1

| | Percent of applied oligosaccharide | | | | |
|---|---|---|---|---|---|
| HS oligosaccharide | NaCl (M) | | | | |
| (Monosaccharide units) | 0.14 | 0.3 | 0.6 | 1.0 | 1.7 |
| 6 | 8 | 44 | 47 | 0.5 | 0.1 |
| 8 | 23 | 21 | 52 | 4 | 0.6 |
| 12 | 17 | 33 | 35 | 13 | 2 |

What is claimed is:

1. A method of modulating the activity of a FGF receptor in a mammal, comprising providing the animal with substantially purified HS, wherein the substantially purified HS has high affinity for FGF7.

2. The method of claim 1, wherein the mammal is a human.

3. The method of claim 1, wherein the FGF receptor is the FGF2IIIb receptor.

4. The method of claim 1, wherein the modulating is antagonistic.

5. The method of claim 1, wherein the modulating is agonistic.

6. The method of claim 1, wherein the substantially purified HS binds to FGF7 in a medium that is about 0.6 M or greater in NaCl.

7. The method of claim 1, wherein the substantially purified HS is a fraction of crude heparin, heparan sulfate or derivatives thereof.

8. The method of claim 1, wherein the substantially purified HS is a fraction of low molecular weight heparin (LMWH) or oligosaccharides.

9. The method of claim 1, wherein the substantially purified HS is synthetic or semi-synthetic HS.

10. The method of claim 1, wherein the substantially purified HS has about four to about twenty saccharide units.

11. The method of claim 10, wherein the substantially purified HS has eight saccharide units.

12. The method of claim 11, wherein the substantially purified HS has 7 or 8 sulfates.

13. The method of claim 12, wherein the substantially purified HS has predominant disaccharide composition of ΔHexA2SGlcN6S and a tri-sulfated disaccharide.

14. The method of claim 1, wherein the substantially purified HS comprises greater than eight saccharide units and contains a motif comprising eight saccharide units, said motif containing 7 or 8 sulfates, wherein said motif has high affinity for FGF7.

15. The method of claim 14, wherein said motif has predominant disaccharide composition of ΔHexA2SGlcN6S and a tri-sulfated disaccharide.

16. The method of claim 1, wherein providing the animal with said substantially purified HS promotes the formation of a ternary complex comprising FGF2IIIb receptor, HS, and FGF in the animal.

17. The method of claim 1, wherein the substantially purified HS is provided via topical administration.

18. The method of claim 17, wherein said HS is dispersed in a pharmaceutically acceptable liquid or solid carrier.

19. The method of claim 18, wherein the pharmaceutically acceptable formulation comprises a wound covering selected from the group consisting of a collagen based cream, a collagen based film, a collagen based microcapsule, a collagen based powder, hyaluronic acid, glycosaminoglycans, creams, foams, an absorption-enhancing formula, suture material, and wound dressings.

20. The method of claim 1, further comprising providing the animal with a FGF.

21. The method of claim 20, wherein the animal is simultaneously provided with substantially purified HS and a FGF.

22. The method of claim 21, wherein the HS and FGF is dispersed in a pharmaceutically acceptable liquid or solid carrier.

23. The method of claim 20, wherein the FGF is FGF7.

24. The method of claim 1, wherein the substantially purified HS has high anticoagulant activity.

25. A composition useful for modulating a FGF receptor in a mammal, comprising FGF7 and substantially purified HS, wherein the substantially purified HS has high affinity for FGF7.

26. A method for obtaining substantially purified HS that modulates a FGF receptor in a mammal, the method comprising:
obtaining an affinity matrix comprising a fibroblast growth factor that preferentially binds to HS that modulates a FGF receptor in a mammal,
contacting the affinity matrix with a mixture comprising heparin, heparan sulfate, oligosaccharides or derivatives thereof,
separating the non-bound material from the bound material, and
obtaining substantially purified HS as the bound material.
